(19) United States
(12) Patent Application Publication
(10) Pub. No.: US 2014/0099292 A1
(43) Pub. Date: Apr. 10, 2014

(54) CD14+ Cell Compositions and Methods of Using Same

(71) Applicant: Aastrom Biosciences, Inc., Ann Arbor, MI (US)

(72) Inventors: Kelly Ledford, Erie, MI (US); Ronnda L. Bartel, San Diego, CA (US); Frank Zeigler, Encinitas, CA (US)

(73) Assignee: Aastrom Biosciences, Inc., Ann Arbor, MI (US)

(21) Appl. No.: 14/037,030

(22) Filed: Sep. 25, 2013

Related U.S. Application Data
(63) Continuation-in-part of application No. PCT/US2013/031241, filed on Mar. 14, 2013.
(60) Provisional application No. 61/614,981, filed on Mar. 23, 2012.

(51) Int. Cl. C12N 5/0786

(52) U.S. Cl. CPC C12N 5/0645
USPC 424/93.71; 435/375; 435/366

(57) ABSTRACT

The present invention provides CD 14⁺ cell compositions and methods of using same in treating disorders, such as inflammatory disorders, such as atherosclerosis and cardiovascular disease.

### RELATED APPLICATIONS

This application is a continuation-in-part of International Application No. PCT/US2013/031241, filed Mar. 14, 2013, which claims the benefit of, and priority to, U.S. Ser. No. 61/614,981, filed Mar. 23, 2012, the contents of which are herein incorporated by reference in their entireties.

### FIELD OF THE INVENTION

The present invention relates generally to compositions of CD14⁺ monocytes and macrophages and their use in treating disorders such as inflammatory disorders, such as atherosclerosis and cardiovascular disease.

### BACKGROUND OF THE INVENTION

Advanced atherosclerotic lesions are characterized by lipid accumulation, chronic inflammation, and defective efferocytosis, all characteristics associated with pro-inflammatory macrophages; therefore it might be beneficial to treat with alternatively activated macrophages where they may facilitate tissue repair.

Thus, a need exists for the identification a suitable source for the in vitro production of alternatively activated macrophages.

### SUMMARY OF THE INVENTION

The present invention is based in part upon the discovery that CD14⁺ hematopoietic cells can be expanded in vitro and differentiated in vitro into CD14⁺ macrophages.

In one aspect the invention provides a composition comprising a population of cells of hematopoietic lineage. For example, the composition is anti-inflammatory. In one embodiment, the composition is anti-atherosclerotic. The composition contains CD14⁺ macrophages and when the cells are contacted with a pro-inflammatory stimulus produce inflammatory cytokines such that the anti-inflammatory cytokine: pro-inflammatory cytokine ratio produced is at least 2:1, or preferably at least 5:1, 10:1, 25:1, 50:1 or 100:1. The population of cells of hematopoietic lineage cells can be derived from bone marrow, peripheral blood, umbilical cord blood, fetal liver, human embryonic stem cells (huES), induce pluripotent stem cells (iPS) or parthenogenetic cells. The CD14+ macrophages can be derived from CD34⁺ hematopoietic progenitor cells that have been differentiated in vitro. Preferably, the CD34⁺ hematopoietic progenitor cells are myeloid cells. More preferably, the myeloid cells are myeolomonocytes.

The composition of the present invention may further contain CD14⁺ monocytes. The CD14⁺ monocytes can be expanded in vitro. The CD14⁺ monocytes can also differentiate into CD14⁺ macrophages in vitro.

The composition of the present invention has one or more of the following characteristics: a) the viability of the cells is at least 75%; b) contains less than 2 μg/ml serum albumin; c) substantially free of horse serum or d) substantially free of mycoplasm, endotoxin and microbial contamination.

The cells of the composition of the present invention are provided in a pharmaceutical-grade electrolyte solution suitable for human administration. Preferably, the total number of cells in the present composition is 40-200 million. Alternatively, the cells of the present composition are in a volume less than 15 mLs. The cells produce at least 100 pg per 2×10⁶ cells of one or more anti-inflammatory cytokines. The anti-inflammatory cytokine produced by the cells may be IL-10 or ILRa. The pro-inflammatory stimulus can be lipopolysaccharide (LPS). Preferably, at least 5% of the CD14⁺ macrophages of the present composition are autofluorescent (auto).

The composition of the present invention can be an in-vitro expanded cell population. Alternatively, the cells of the instant composition are isolated from an in-vitro expanded cell culture. Preferably, the in-vitro expanded cell culture is derived from mononuclear cells. In some embodiment, the in-vitro expanded cell culture contains a mixed population of cells of hematopoietic, mesenchymal and endothelial linage. In some embodiment, the in-vitro expanded cell culture contains a mixed population of cells of hematopoietic and mesenchymal linage. In another embodiment, the in-vitro expanded cell culture contains a population of hematopoietic cells. For example, the mixed population of cells is about 5-75% viable CD90⁺ cells with the remaining cells in the composition being CD45⁺. For example, the hematopoietic cells are CD45⁺.

In one aspect, at least 5% or at least 10% of the CD14⁺ macrophages of the cell composition are CD66b-negative, CD18⁺, CD33⁺, CD11b⁺, CD11c⁺, CD91-negative, CD141⁺, HLA-DR-negative, CD209-negative, CD16-negative, and/or CD1c-negative.

In another aspect, at least 15%, 20%, 25%, 30%, 40%, 50%, 60%, 70%, 80%, 90%, 95%, 98%, or greater, of the CD14⁺ macrophages of the cell composition are CD66b-negative, CD18⁺, CD33⁺, CD11b⁺, CD91-negative, CD141⁺, HLA-DR-negative, CD209-negative, CD16-negative, and/or CD1c-negative.

In another aspect, the CD14⁺ macrophages express PPARγ, CD206, CD163, CD204, SR-B1, MERTK, and/or TGFβ. For example, at least 5%, 10%, 15%, 20%, 30%, 40%, 50%, 60%, 70%, 80%, 90%, 95%, 98%, or greater of the CD14⁺ macrophages express PPARγ, CD206, CD163, CD204, SR-B1, MERTK, and/or TGFβ.

In another aspect, the CD 14⁺ macrophages express a higher level of PPARγ, CD206, CD163, CD204, SR-B1, MERTK, and/or TGFβ compared to M1 macrophages. For example, the CD14⁺ macrophages express an at least 2-fold, 3-fold, 4-fold, 5-fold, 8-fold, 10-fold, 15-fold, 20-fold, 50-fold, 100-fold, 250-fold, 500-fold, or higher level of PPARγ, CD206, CD163, CD204, SR-B1, MERTK, and/or TGFβ compared to M1 macrophages.

In another aspect, the CD14⁺ macrophages express a lower level of CCR7, IL-1B, and/or TNFα compared to M1 macrophages. For example, the CD14⁺ macrophages express an at least 2-fold, 3-fold, 4-fold, 5-fold, 8-fold, 10-fold, 15-fold, 20-fold, 50-fold, 100-fold, 250-fold, or 500-fold lower level of CCR7, IL-1B, and/or TNFα compared to M1 macrophages.

In another aspect, the CD14⁺ macrophages do not significantly increase their expression of pro-inflammatory cytokines after exposure to a pro-inflammatory stimulus. For example, the expression level of a pro-inflammatory cytokine in the CD14⁺ macrophages after exposure to a pro-inflammatory stimulus is 300%, 200%, 100%, 50%, 40%, 30%, 20%, 10%, 5%, 3%, 2%, 1%, 0.1%, 0.001%, or less of the expression level of the pro-inflammatory cytokine in prior to exposure to the pro-inflammatory stimulus.

Exemplary pro-inflammatory stimuli include but are not limited to pathogens (e.g., bacteria, viruses, or protozoa), lipopolysaccharide (LPS), interferon gamma (IFN-γ), lipoxin, leukotriene, endotoxin, and debris from dead cells.

Exemplary pro-inflammatory cytokines include but are not limited to TNFα, IL-1A, IL-1B, and IL-12.

Methods generally available in the art are used to determine the expression level of a protein described herein. For example, the expression level refers to the level of a protein (e.g., secreted protein or cell surface marker) described herein. In other embodiments, the expression level refers to the level of a nucleic acid that encodes a protein (e.g., secreted protein or cell surface marker) described herein.

Also provided herein are methods of modulating cholesterol efflux in vascular tissue of a subject by administering to a subject in need thereof any composition of the present invention or a composition containing ixmyelocel-T.

Another aspect of the invention is methods of decreasing atherosclerotic lesions in a subject by administering to a subject in need thereof any composition of the present invention or a composition containing ixmyelocel-T.

A further aspect of the invention is methods of treating atherosclerosis by administering to a subject in need thereof the composition of any composition of the present invention or a composition containing ixmyelocel-T.

Also provided are methods of decreasing oxidative stress of a tissue by contacting the tissue with any composition of the present invention or a composition containing ixmyelocel-T. Preferably, the tissue is endothelium.

The present invention further provides methods of increasing plasma nitrate levels and/or decreasing plasma lipid peroxidation in a subject by administering to a subject in need thereof any composition of the present invention or a composition comprising ixmyelocel-T.

Also included in the invention are methods of increasing the expression of endothelial nitric oxide synthase (eNOS) and/or nitric oxide production (NO) in a cell by contacting the cell with any composition of the present invention or a composition comprising ixmyelocel-T.

In another aspect, the invention includes methods of tissue regeneration or repair by administering a patient in need thereof any composition of the present invention.

The invention is also directed to method of treating ischemic disorders by administering a patient a composition comprising a population of cells of hematopoietic lineage. The composition contains CD14⁺ macrophages and when the cells are contacted with a pro-inflammatory stimulus produce inflammatory cytokines such that the anti-inflammatory cytokine: pro-inflammatory cytokine ratio produced is at least 2:1.

In yet a further aspect, the invention provides methods of inducing angiogenesis in a tissue comprising administering a patient a composition comprising a population of cells of hematopoietic lineage. The composition contains CD14⁺ macrophages and when the cells are contacted with a pro-inflammatory stimulus produce inflammatory cytokines such that the anti-inflammatory cytokine: pro-inflammatory cytokine ratio produced is at least 2:1.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### BRIEF DESCRIPTION OF THE DRAWINGS

FIG. 1 is an illustration of atherosclerosis development and complications, including critical limb ischemia and ischemic dilated cardiomyopathy.

FIG. 2 is an illustration showing that atherosclerosis is a multi-factorial disease of the vessel wall (adapted from Libby P. Nature 420, 868-874, 2002, the contents of which are incorporated herein by reference).

FIG. 3 is an illustration depicting the role of macrophages in atherosclerosis.

FIG. 4 is an illustration depicting the processes involved in maintenance of macrophage cholesterol homeostasis.

FIG. 5 is an illustration depicting reverse cholesterol transport (RCT).

FIG. 6A-B are illustrations depicting cholesterol efflux from a macrophage.

FIG. 7 is an illustration of the in vitro expansion of the CD14⁺ cell compositions of the invention.

FIG. 8 is a series of histograms showing PKH proliferation analysis of the phenotypes in ixmyelocel-T.

FIG. 9 is a panel of images showing surface expression of two well-characterized markers of alternatively activated macrophages, CD206 and CD163, on C14⁺ ixmyelocel-T macrophages of the invention.

FIG. 10 is a bar graph showing the expression on CD14⁺ ixmyelocel-T macrophages of the invention of several scavenger receptors reported to take up modified cholesterol and apoptotic cells.

FIG. 11 is a panel of flow cytometry scatterplots showing the CD66b and CD18 phenotypes of the CD14⁺ cells of the invention. The top plots are the isotype controls.

FIG. 12 is a panel of flow cytometry scatterplots showing the CD33 and CD11b phenotypes of the CD14⁺ cells of the invention. The top plots are the isotype controls.

FIG. 13 is a panel of flow cytometry scatterplots showing the CD11c and CD91 phenotypes of the CD14⁺ cells of the invention. The top plots are the isotype controls.

FIG. 14 is a panel of flow cytometry scatterplots showing the CD141 and HLA-DR phenotypes of the CD14⁺ cells of the invention. The top plots are the isotype controls.

FIG. 15 is a panel of flow cytometry scatterplots showing the CD209 and CD1c phenotypes of the CD14⁺ cells of the invention. The top plots are the isotype controls.

FIG. 16 is a bar graph showing the levels of anti-inflammatory cytokines. IL-10, IL-r1a, TNFα, IL-10, and IL-12 were quantified in MACS sorted CD14⁺ sorted ixmyelocel-T supernatants treated with and without LPS (n>3). Ixmyelocel-T macrophages secrete elevated levels of anti-inflammatory cytokines, before and after LPS stimulation, while pro-inflammatory cytokine secretion remains minimal. * P<0.05 vs. basal,** P<0.001 vs. basal.

FIG. 17 is a series of bar graphs showing cytokine levels after ixmyelocel-T macrophages are loaded with oxidized LDL and are subjected to LPS challenge.

FIG. 18 is a chart showing the quantification of cytokines in supernatants from modified cholesterol loaded ixmyelocel-T macrophages and THP-1 macrophages treated with and without LPS (n≧6). The amount of cytokine expressed was normalized to the total protein concentration of each sample. Values are presented as mean±SEM relative to control, *p<0.05, **p<0.01, ***p<0.001 vs. THP-1−LPS; #p<0.05, ## p<0.001 vs. IXT−LPS, $p<0.001 vs. THP-1+LPS.

FIG. 19 is a series of bar graphs showing the expression level of genes involved in cholesterol efflux.

FIG. 20A is a series of fluorescent microscopy images of ixmyelocel-T macrophages and THP-1 macrophages loaded with Dil-Ac-LDL. Magnification: 40×. FIG. 20B is a set of bar graphs showing quantitative real-time PCR gene expression analysis of scavenger receptors normalized to GAPDH, the relative control (n>5). Expression of CD36 and SCARB1 in THP-1 and ixmyelocel-T macrophages before and after lipid loading is shown. Values are presented as mean±SEM relative to control, *p<0.01 vs. THP-1−Ac-LDL, **p<0.001 vs. THP-1−Ac-LDL.

FIG. 21 is a schematic depicting cholesterol influx and efflux pathways and a series of bar graphs showing expression of cholesterol transport genes. Quantitative real-time PCR gene expression analysis is shown of scavenger receptors normalized to GAPDH, the relative control (n≧5). Expression of ABCA1, ABCG1, ACAT1, and CEH in THP-1 and ixmyelocel-T macrophages before and after lipid loading was analyzed. Values are presented as mean±SEM relative to control, *p<0.05, ** p<0.01, *** p<0.001 vs. THP-1−Ac-LDL; #p<0.05, ##p<0.01 vs. IXT−Ac-LDL.

FIG. 22 is a bar graph showing level of cholesterol efflux. The ability of ixmyelocel-T macrophages to efflux cholesterol was measured with an in vitro cholesterol efflux assay. Ixmyelocel-T macrophages and THP-1 macrophages were loaded with free cholesterol using radiolabeled acetylated LDL (³H-cholesterol-AcLDL). Ixmyelocel-T macrophages demonstrated a robust increase in ABCA1-mediated cholesterol eflux, as seen by the increase in efflux to apoA-I. (n=4) * p<0.01, **p<0.001 vs. THP-1.

FIG. 23 is a line graph (A), set of bar graphs (B), and schematic (C) showing in vivo cholesterol efflux examined in scid mice after intraperitoneal injections of either ³H-cholesterol-loaded J774 cells or ixmyelocel-T macrophages. Plasma ³H-cholesterol levels were determined after 24 and 48 hours, ³H-tracer found in the liver, and ³H-tracer found in the feces after 48 hours (n>3 per group). Values are presented as mean±SEM relative to control, *p<0.05 vs. J774.

FIG. 24A is a series of images showing the co-localization of TRCs and eNOS. FIG. 24B-C is a set of bar graphs showing the effect of ixmyelocel-T treatment on plasma nitrates and TBARS.

FIG. 25A is a set of immunofluorescence images showing expression of eNOS in HUVECs co-cultured with ixmyelocel-T or BMMNCs. FIG. 25B is a set of bar graphs showing the expression of eNOS measured by ELISA in HUVECs co-cultured with ixmyelocel-T or BMMNCs.

FIG. 26 is a set of bar graphs showing the levels of NO and nitrates produced by HUVECs co-cultured with ixmyelocel-T or BMMNCs.

FIG. 27 is a set of bar graphs showing intracellular ROS levels in TNFα and oxidized LDL-stimulated HUVECs co-cultured with ixmyelocel-T.

FIG. 28 is a set of bar graphs showing the levels of ROS and the SOD activity in HUVECs co-cultured with ixmyelocel-T or BMMNCs.

FIG. 29 is a set of bar graphs showing the effect of ixmyelocel-T or BMMNCs on viability and apoptosis in TNFα treated HUVECs.

FIG. 30A is a bar graph showing the percentage of apoptotic cells with ixmyelocel-T macrophages. FIG. 30B is a set of microscopy images showing localization of apoptotic cells and ixmyelocel-T macrophages. FIG. 30C is a set of flow cytometry plots showing efferocytosis.

FIG. 31 is a series of bar graphs depicting the relative expression levels of adhesion molecules in HUVECs with and without co-culture with ixmyelocel-T, and with and without TNFα.

FIG. 32 is a series of bar graphs depicting the expression levels of MCP-1 in HUVECs with and without co-culture with ixmyelocel-T, and with and without TNFα.

FIG. 33 is a bar graph depicting the level of IL-10 secreted by HUVECs with and without co-culture with ixmyelocel-T, and with and without TNFα.

FIG. 34A is a set of flow cytometry plots showing CD16 and CD14 expression on ixmyelocel-T macrophages. The cells were stained for the CD16 marker.

FIG. 34B is a set of flow cytometry plots showing HLA-DR and CD14 expression on ixmyelocel-T macrophages. The cells were stained for the HLA-DR marker.

FIGS. 35A-J are a set of bar graphs showing expression of M1 and M2 macrophage cell markers on M1, M2, and Ix-MAC macrophage cells. Quantitative real-time PCR gene expression analysis was performed, normalized to GAPDH (n≧4). The relative expression of (A) peroxisome proliferator-activated receptor gamma (PPARγ), (B) CD206, (C) CD163, (D) CD204, (E) Scavenger receptor class B member 1 (SR-B1), (F) MER Receptor Tyrosine Kinase (MERTK), (G) Chemokine (C-C Motif) Receptor 71 (CCR7), (H) TNFα, (I) IL-1β, and (J) and transforming growth factor beta (TGFβ) in M1, M2, and ixmyelocel-T macrophages (IXT) are plotted. Values are presented as mean±SEM relative to IXT, ^{*}P<0.05, ^{**}P<0.01, ^{***}P<0.001 vs M1, ^{#}P<0.05, ^{##}P<0.01, ^{###}P<0.001 vs M2.

FIGS. 36A-B are a set of bar graphs showing pro-inflammatory cytokine expression in M1, M2,and Ix-MAC macrophage cells (IXT) after inflammatory challenge. (A) TNFα and (B) IL-12 were quantified in M1, M2, and IxMAC supernatants treated with and without 0.1 μg/mL LPS (n=4-7). Values are presented as mean ±SEM relative to control, ^{*}P<0.001 vs M1. ^{#}P<0.001 vs M2.^{&}P<0.05 vs IXT. ^{̂}P <0.05, ^{̂̂}P<0.001 vs M1+LPS. ^{%}P<0.001 vs M2+LPS. SEM=standard error of the mean.

### DETAILED DESCRIPTION OF THE INVENTION

Cells of the Invention

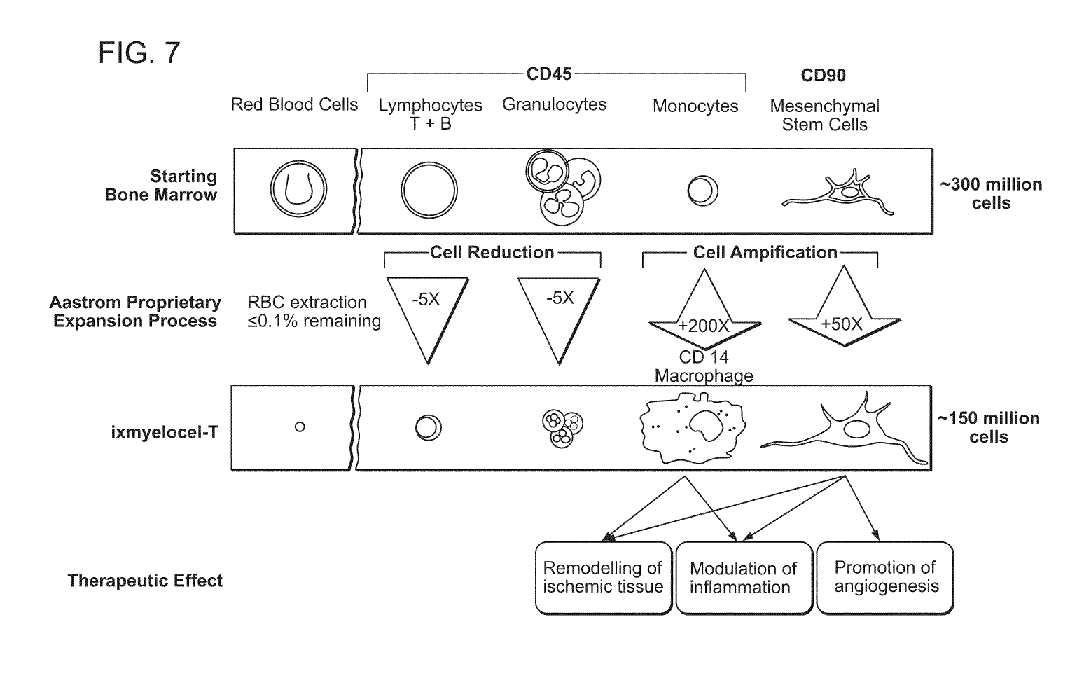

The invention is based in part upon the discovery that CD 14⁺ hematopoietic cells can be expanded in vitro and differentiated in vitro into CD 14⁺ macrophages. More surprisingly, this in vitro expanded CD 14⁺ macrophage cell population upregulates the expression of anti-inflammatory cytokine expression when stimulated with a pro-inflammatory stimulus. The in vitro expanded CD14⁺ myelomonocyte/macrophage cell population was originally discovered as a subpopulation of cells in Tissue Repair Cells (TRCs) also know as ixmyelocel-T. Isolation, purification, characterization, and culture of TRCs is described in WO/2008/054825, the contents of which are incorporated by reference its entirety. The in vitro expanded CD14⁺ macrophage cell population of the invention are referred to herein as “Ix-MACs” (FIG. 7).

Ix-MACs contain CD le macrophages of hematopoietic cell lineage produced from mononuclear cells. Optionally, Ix-MACs also contain CD14⁺ monocytes. At least 5%, 10%, 15%, 20%, 25%, 30%, 35%, 40%, 45%, 50% or more of the CD14⁺ macrophages are CD14^{+auto} (autofluorescent). The mononuclear cells are isolated from adult, juvenile, fetal or embryonic tissues. For example, the mononuclear cells are derived from bone marrow, peripheral blood, umbilical cord blood fetal liver tissue, human embroyonic stem cells (huES), induce pluripotent stem cells (iPS), or parthenogenetic cells

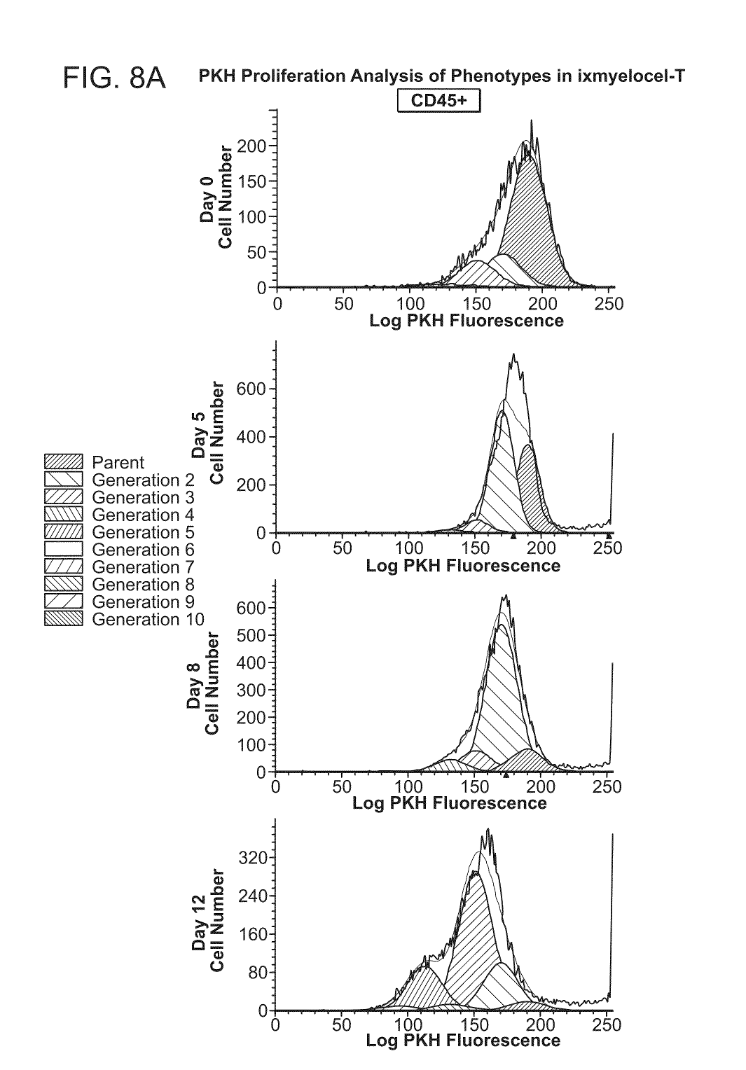

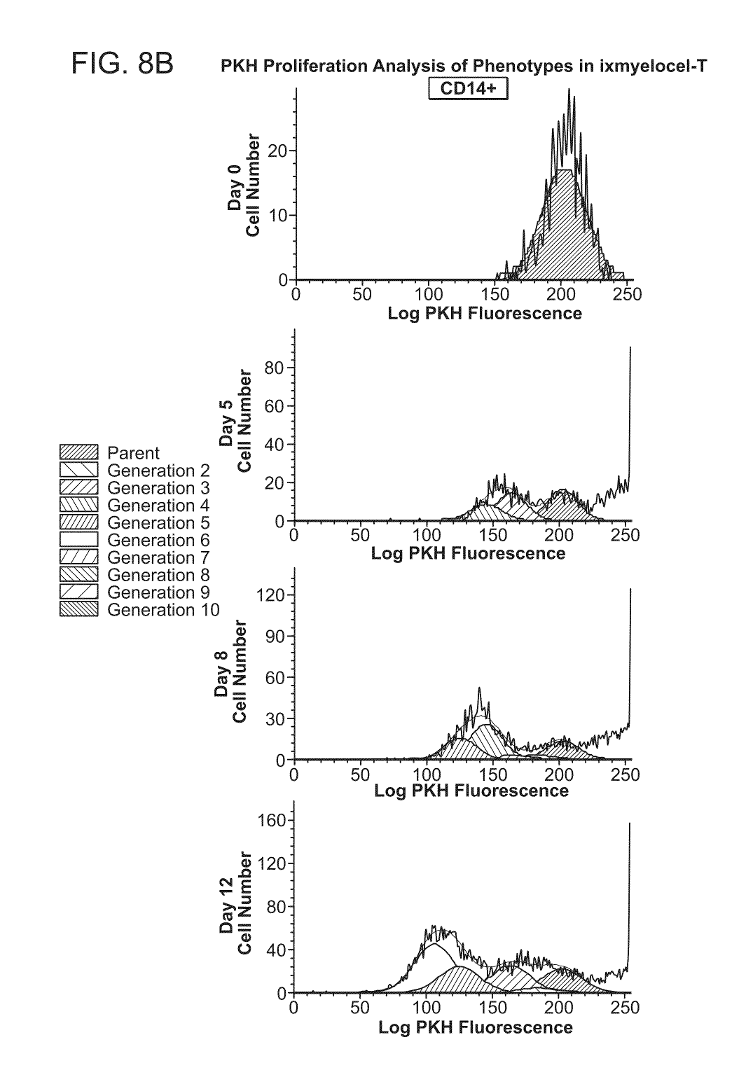

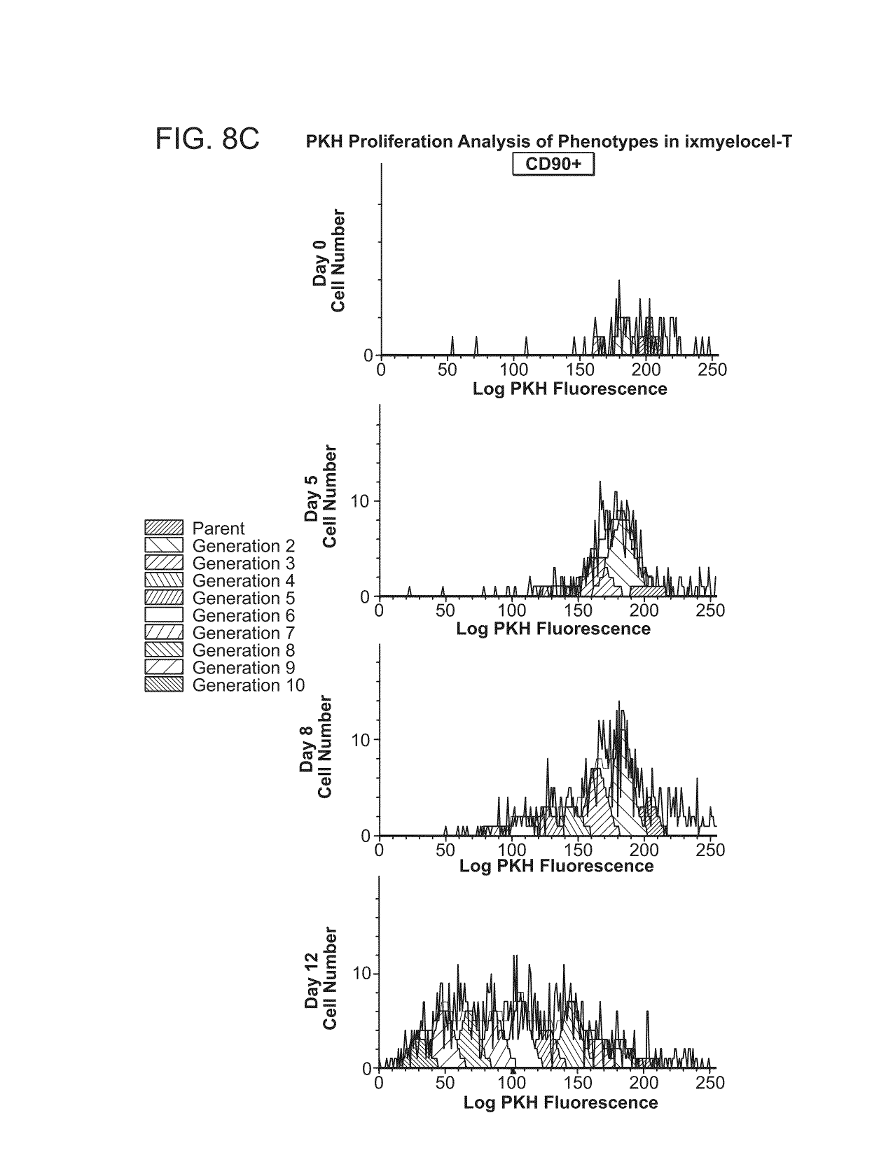

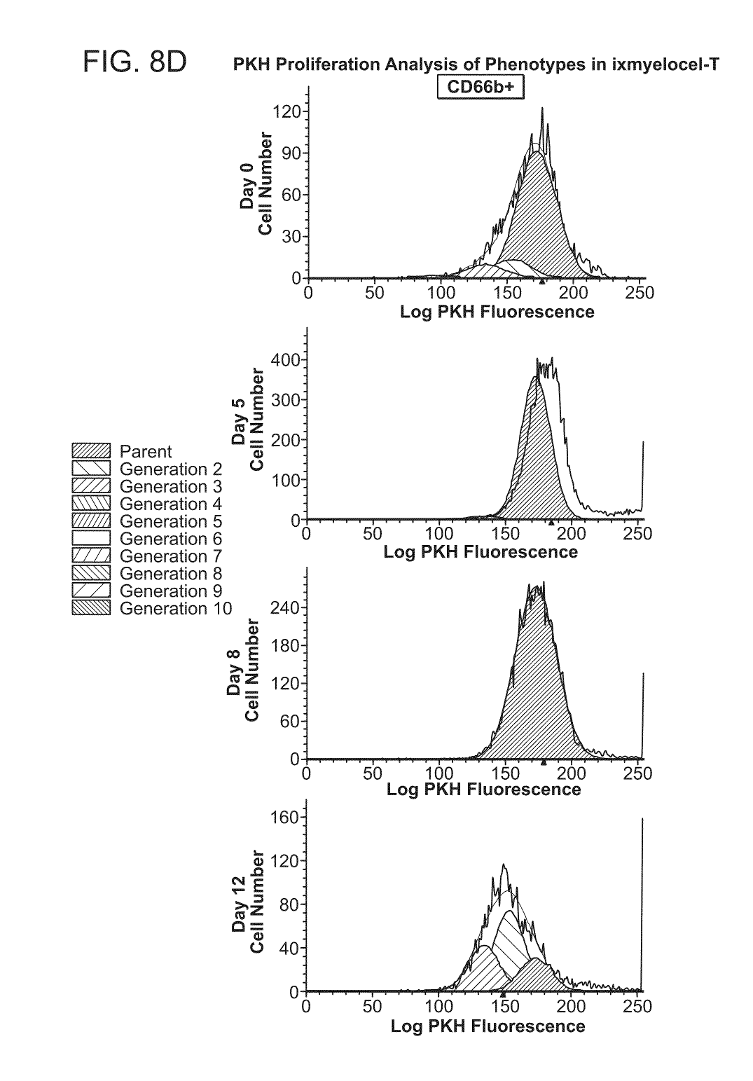

The CD 14⁺ macrophages are derived from in vitro expanded CD14⁺ myelomonocytes that have differentiated into macrophages in vitro. FIG. 8 shows the in vitro proliferation of the CD14⁺ cells.

Ix-MACs are produced, for example by an in vitro culture process that results in a unique cell composition. Additionally, the Ix-MACs of the instant invention have both high viability and low residual levels of components used during their production.

The CD14⁺ cells in ixmyelocel-T (Ix-MACs) are generated from a combination of direct differentiation with little or no expansion from monocytes (constituting a majority, i.e., about 75%, of the Ix-MACs) and to a lesser extent through limited proliferation of monocytes/myeloid progenitors (constituting a minority, i.e., about 25% or less).

In some aspects, the cell compositions of the invention consist essentially of CD14⁺ macrophages of hematopoietic lineage. For example, the cell compositions of the invention are made up of at least 50%, 60%, 70%, 80%, 85%, 90%, 95%, 98%, 99%, or greater, CD14⁺ macrophages of hematopoietic lineage. For example, at least 5%, 10%, 20%, 25%, 30%, 35%, 40%, 45%, 50%, 55%, 60%, 65%, 70%, 75%, 80%, 85%, 90%, 95%, 98%, or greater, of the CD14⁺ macrophages are autofluorescent.

In some aspects, at least 26%, 27%, 28%, 29%, 30%, 35%, 40%, 50%, 60%, 70%, 80%, 90%, 92%, 93%, 94%, 95%, 96%, 97%, 98%, 99%, or greater, of the cells of the compositions of the invention are CD14^{+auto} (autofluorescent) cells (e.g., CD14^{+auto} (autofluorescent) macrophages).

The viability of the Ix-MACs is at least 50%, 60%, 70%, 75%, 80%, 85%, 90%, 95% or more. Viability is measured by methods known in the art, such as trypan blue exclusion. This enhanced viability and low residual levels of components makes the Ix-MACs composition highly suitable for human therapeutic administration, as well as enhances the shelf-life and cryopreservation potential of the final cell product.

By components used during production is meant, but not limited to, culture media components such as horse serum, fetal bovine serum and enzyme solutions for cell harvest. Enzyme solutions include trypsins (animal-derived, microbial-derived, or recombinant), various collagenases, alternative microbial-derived enzymes, dissociation agents, general proteases, or mixtures of these. Removal of these components provides for safe administration of Ix-MACs to a subject.

Preferably, the Ix-MACs compositions of the invention contain less than 10, 5, 4, 3, 2, or 1 μg/ml bovine serum albumin; less than 5, 4, 3, 2, 1, 0.9, 0.8, 0.7, 0.6, or 0.5 μg/ml harvest enzymes (as determined by enzymatic activity) and are substantially free of mycoplasm, endotoxin and microbial (e.g., aerobic, anaerobic and fungi) contamination.

By substantially free of endotoxin is meant that there is less endotoxin per dose of Ix-MACs than is allowed by the FDA for a biologic, which is a total endotoxin of 5 EU/kg body weight per day, which for an average 70 kg person is 350 EU per total dose of TRCs.

By substantially free of mycoplasma and microbial contamination is meant as negative readings for the generally accepted tests known to those skilled in the art. For example, mycoplasm contamination is determined by subculturing an Ix-MACs product sample in broth medium and distributed over agar plates on day 1, 3, 7, and 14 at 37° C. with appropriate positive and negative controls. The product sample appearance is compared microscopically, at 100×, to that of the positive and negative control. Additionally, inoculation of an indicator cell culture is incubated for 3 and 5 days and examined at 600× for the presence of mycoplasmas by epifluorescence microscopy using a DNA-binding fluorochrome. The product is considered satisfactory if the agar and/or the broth media procedure and the indicator cell culture procedure show no evidence of mycoplasma contamination.

The sterility test to establish that the product is free of microbial contamination is based on the U.S. Pharmacopedia Direct Transfer Method. This procedure requires that a pre-harvest medium effluent and a pre-concentrated sample be inoculated into a tube containing tryptic soy broth media and fluid thioglycollate media. These tubes are observed periodically for a cloudy appearance (turbidity) for a 14 day incubation. A cloudy appearance on any day in either medium indicate contamination, with a clear appearance (no growth) testing substantially free of contamination.

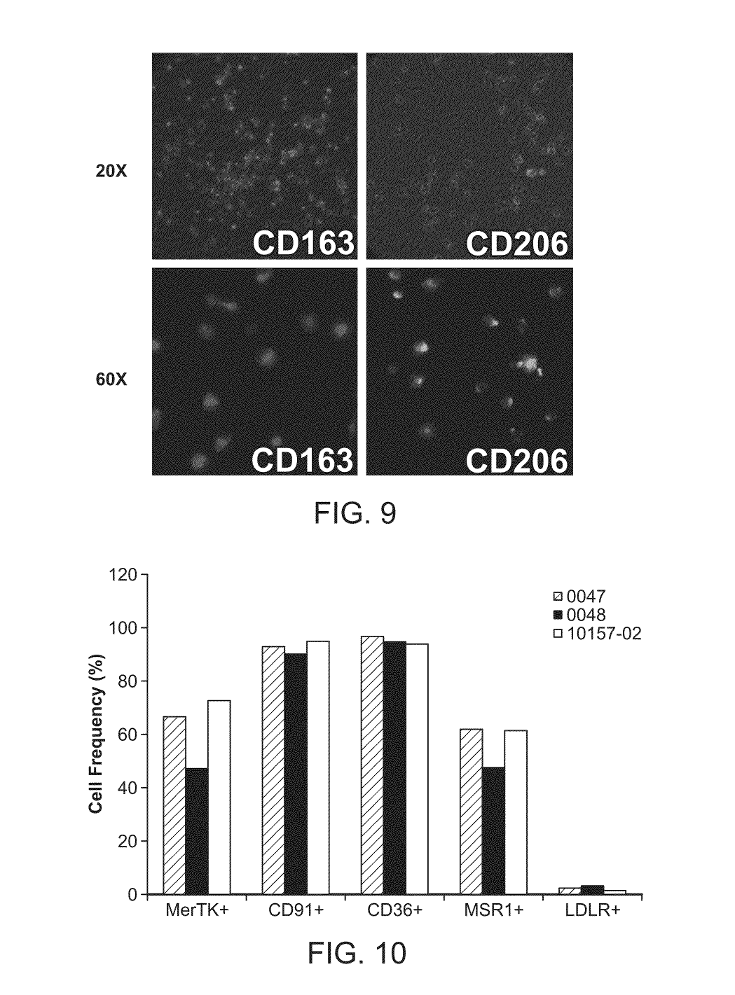

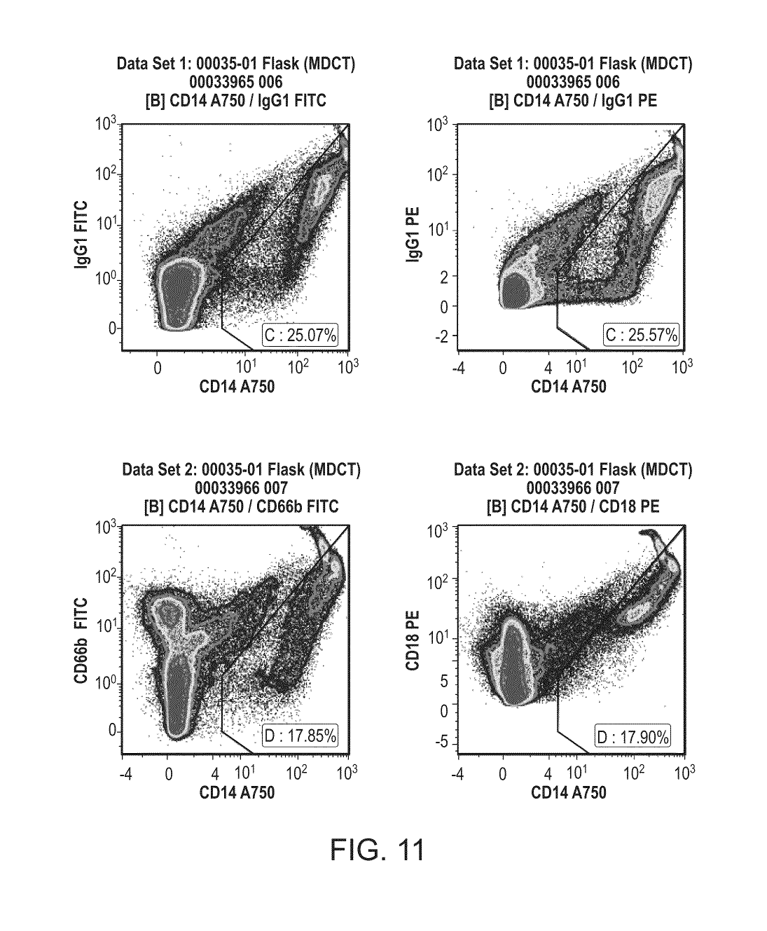

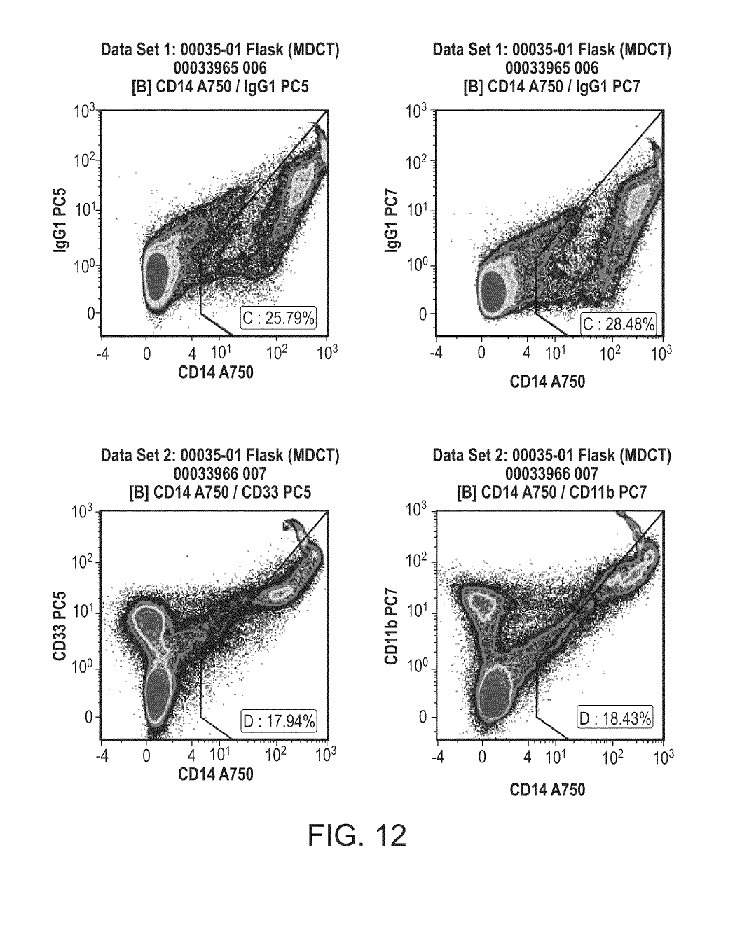

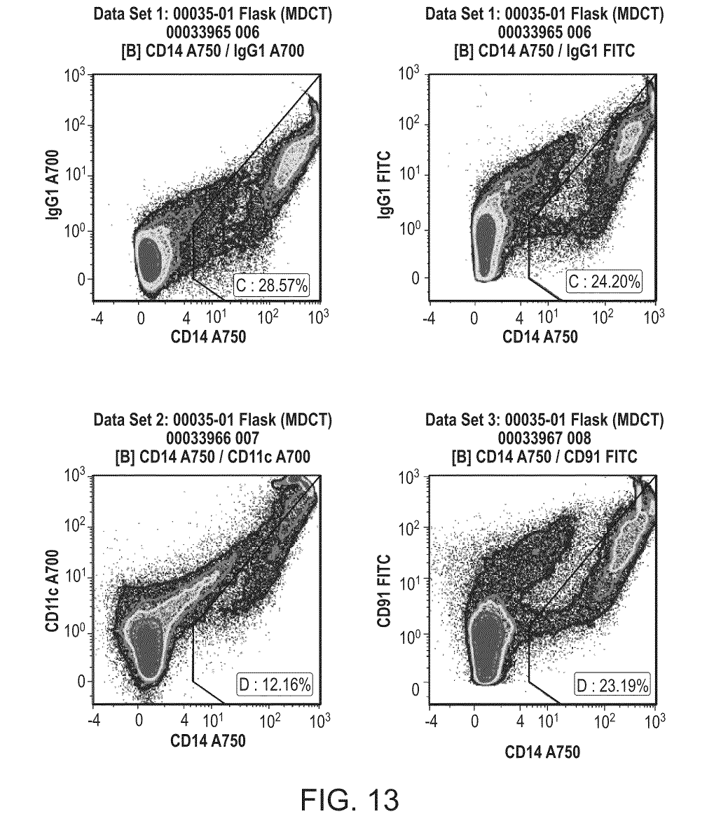

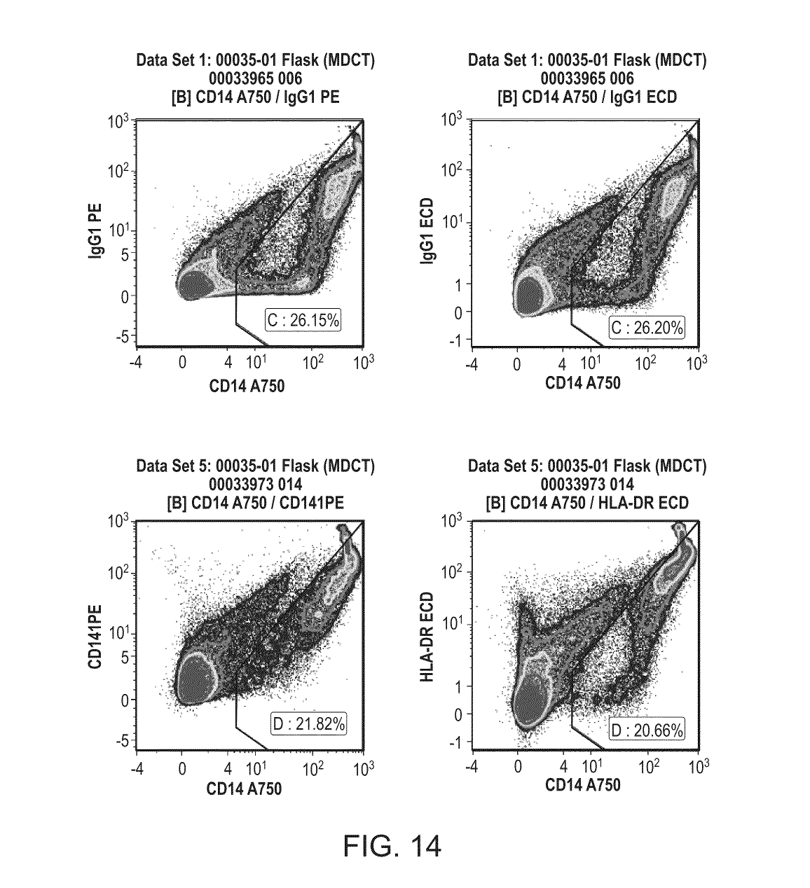

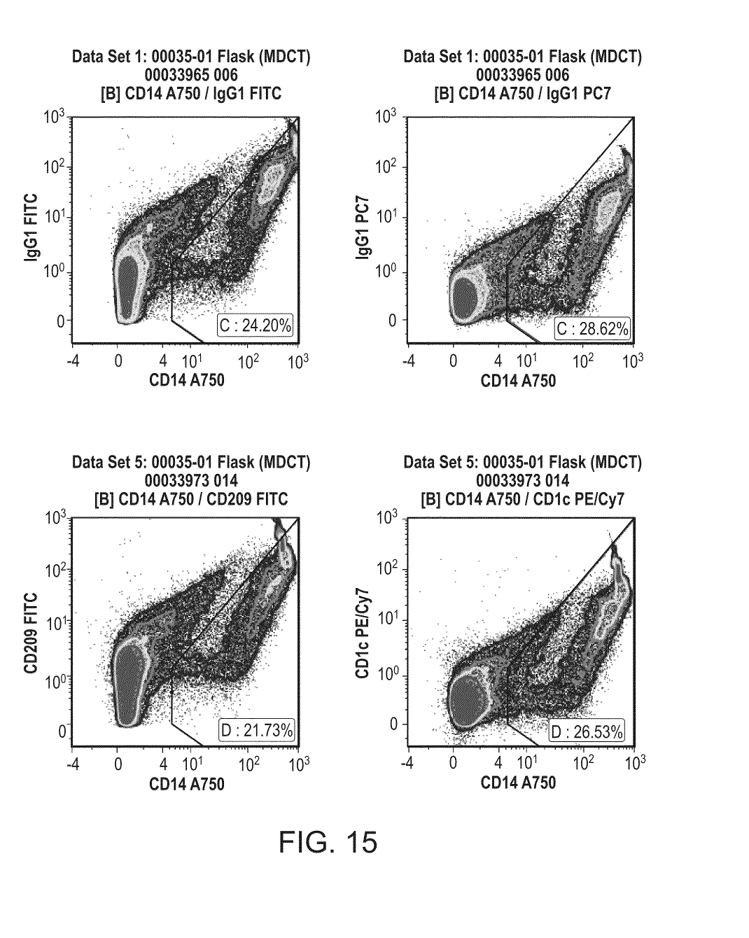

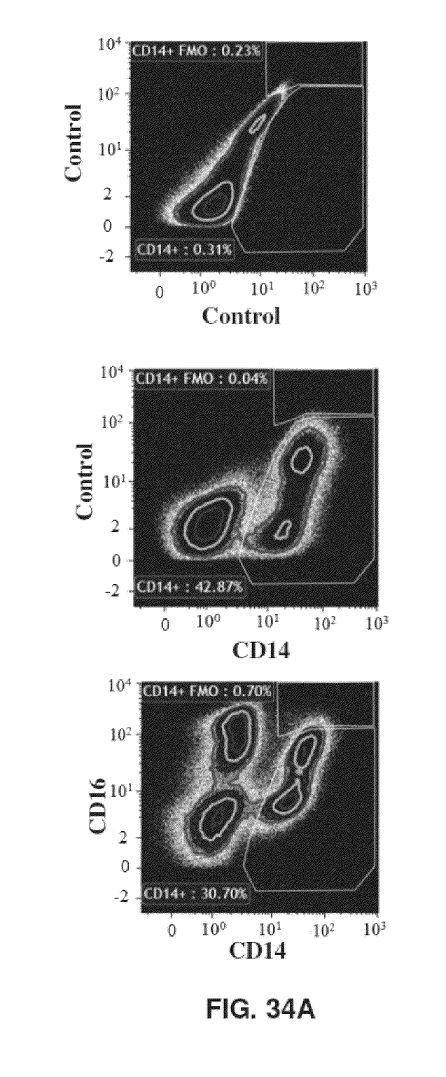

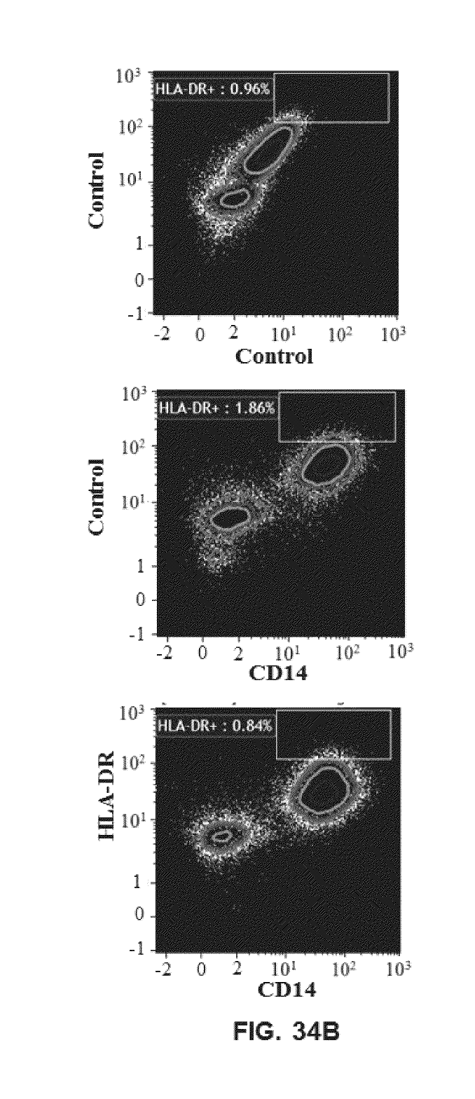

The cells of the Ix-MACs composition have been characterized by cell surface marker expression. As shown in FIG. 9, the Ix-MACs express CD206 and CD163, which are markers of activated macrophages. Additionally, as shown in FIG. 10, the Ix-MACs also express several scavenger receptors such as MerTk, CD91, CD36, MSR1 and LDLR that have been reported to take up modified cholesterol and apoptotic cells. In addition, Ix-MACs do not express pro-inflammatory macrophage surface receptors, e.g., CD16 and HLA-DR, as measured using flow cytometry by staining CD16 or HLA-DR markers (FIGS. 34A-B). Also, flow cytometry was used to perform additional phenotyping of the C14⁺ Ix-MACs. The CD 14⁺ Ix-MACs were CD66b-neg, CD18⁺, CD33⁺, CD11b⁺ (FIGS. 11-12), CD11c⁺, CD91-neg, CD141⁺, HLA-DR-neg (FIGS. 13-14), CD209-neg, and CD1c-neg (FIG. 15).

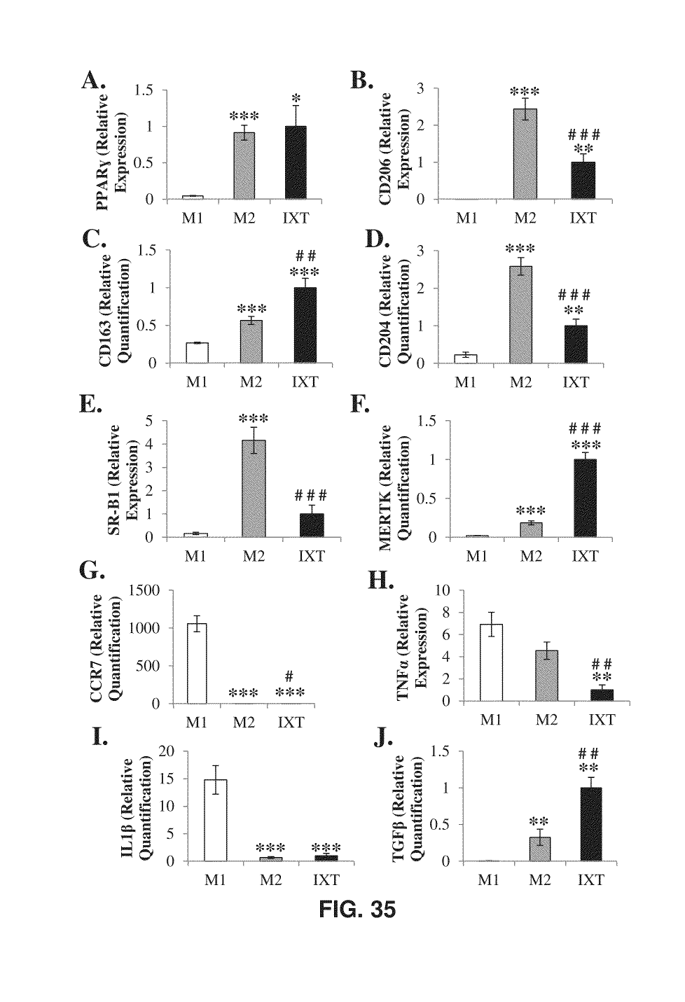

In addition, the cell marker expression profile of Ix-MACs was compared to that of M1 and M2 macrophages. Macrophages are classically divided into two subsets based on their responsiveness to inflammatory stimuli—classically activated (M1) and alternatively activated (M2) macrophages. M1 macrophages are thought to play a role in killing tumor cells and foreign organisms, while M2 macrophages are thought to be involved in wound healing, angiogenesis, and debris scavenging. As shown in FIGS. 35A-J, quantitative real-time PCR gene expression analysis was performed of M1 and M2 macrophage markers within Ix-MACs, M1, and M2 macrophages, and the relative expression levels of peroxisome proliferator-activated receptor gamma (PPARγ), CD206, CD163, CD204, SR-B1, MERTK, CCR7, TNFβ, IL-1β, and transforming growth factor beta (TGFβ) were determined (FIGS. 35A-J). Ix-MACs express PPARγ, CD206, CD163, CD204, SR-B1, MERTK, and TGFβ. For example, Ix-MACs express significantly higher levels of PPARγ, CD206, CD163, CD204, SR-B1, MERTK, and TGFβ than M1 macrophages. Also, Ix-MACs express a significantly lower level of CCR7 than M1 and M2 macrophages, a lower level of IL-1B than M1 macrophages (similar to that of M2 macrophages), and a lower level of TNFα than M1 and M2 macrophages. Thus, Ix-MACs have a similar but not identical cell marker expression profile as M2 macrophages, and Ix-MACs have a different cell marker expression profile from that of M1 macrophages.

Ix-MACs and Markers of Inflammation

Ix-MACs remain anti-inflammatory after pro-inflammatory stimulus. After exposure to a pro-inflammatory stimulus, the Ix-MACs produce inflammatory cytokines. Specifically, after exposure to a pro-inflammatory stimulus, the Ix-MACs upregulate the production of anti-inflammatory cytokines such that the anti-inflammatory cytokine: pro-inflammatory cytokine ratio produced by the Ix-MACs is at least 2:1, 5:1, 10:1, 25:1, 50:1 or 100:1, or more. Anti-inflammatory cytokines include, for example, IL-10 and IL-1ra. Pro-inflammatory cytokines include, for example, TNF alpha, IL-1B, and IL-12.

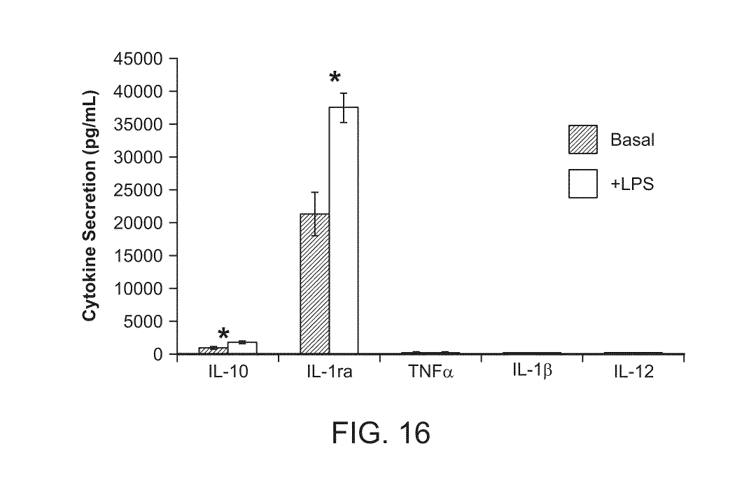

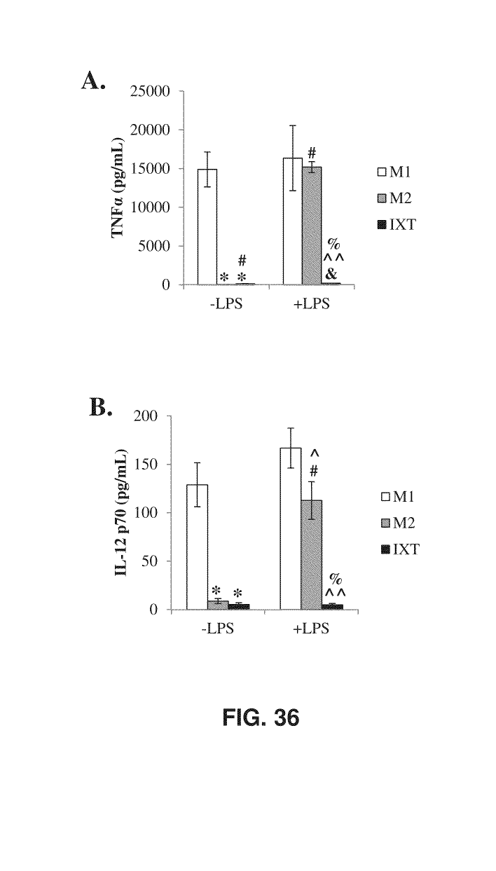

Inflammatory cytokine production of the Ix-MACs composition was determined. As shown in FIG. 16, IL-10, IL-r1a, TNFα, IL-1B, and IL-12 were quantified in Ix-MACs before and after LPS stimulation (i.e., pro-inflammatory stimulus). As demonstrated in FIG. 16, unstimulated Ix-MACs secrete anti-inflammatory cytokines IL-10 and IL-1RA, both of which are upregulated upon pro-inflammatory stimulus. Surprisingly, the secretion level of pro-inflammatory cytokines TNFα, IL-1B and IL-12 are minimal both before and after pro-inflammatory stimulus. Also, secretion of pro-inflammatory cytokines TNFα and IL-12 were compared in Ix-MACs versus M1 and M2 macrophages after inflammatory challenge. In particular, TNFα (FIG. 36A) and IL-12 (FIG. 36B) were quantified in M1, M2, and Ix-MAC supernatants treated with or without 0.1 μg/mL LPS. M1 macrophages secreted TNFα and IL-12 with or without LPS treatment. M2 macrophages secreted more TNFα and IL-12 upon LPS challenge. In contrast, Ix-MACs (IXT) surprisingly did not increase their secretion levels of TNFα or IL-12 after LPS challenge. Thus, Ix-MACs surprisingly remain anti-inflammatory after inflammatory (e.g., LPS) challenge.

Taken together, these results indicate that, while Ix-MACs have similarities to M2 macrophages (which are generally involved in wound healing, angiogenesis, and debris scavenging) in terms of cell marker expression, Ix-MACs also have characteristics that are distinct from both M1 and M2 macrophages.

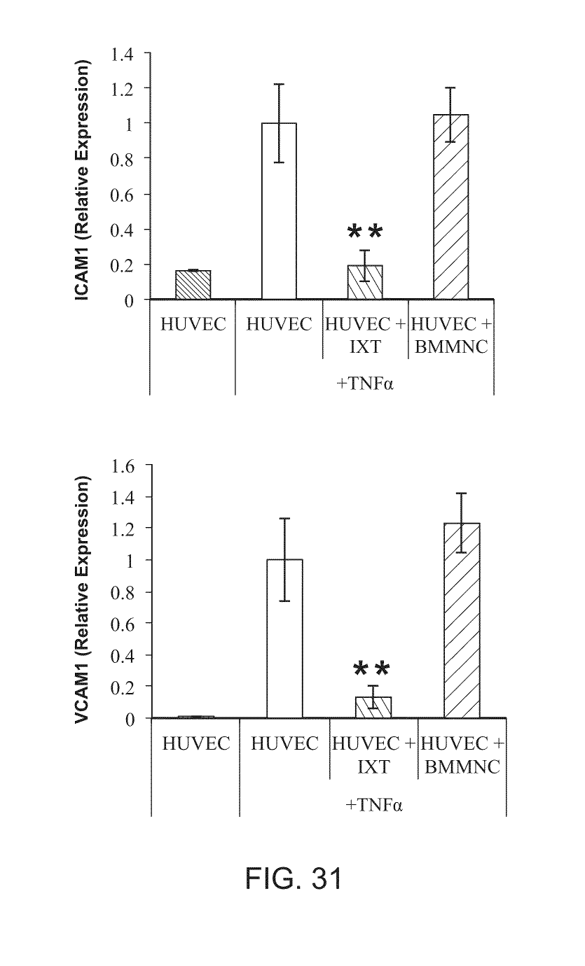

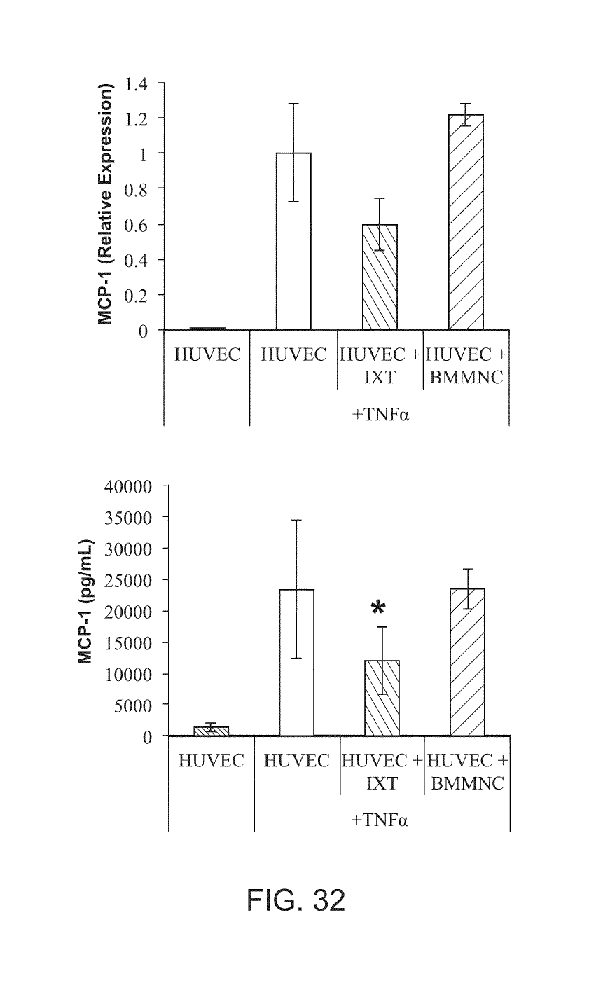

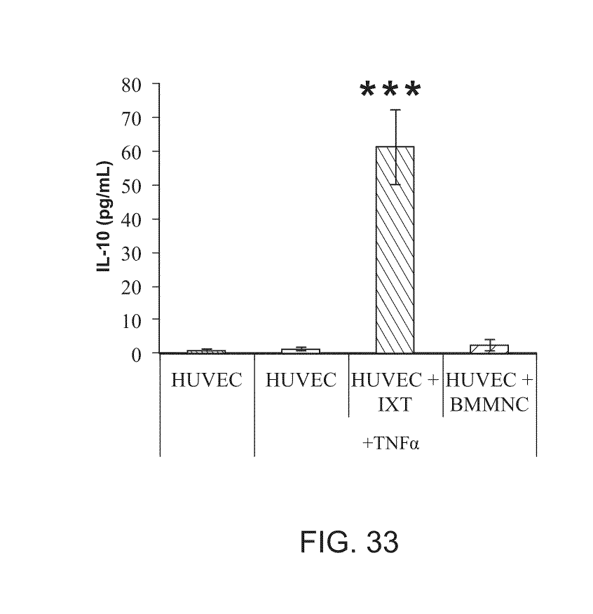

In addition, markers of inflammation were analyzed with RT-PCR in HUVECs that were stimulated with TNFα and co-cultured with ixmyelocel-T or bone marrow derived mononuclear cells (BMMNCs). TNFα treatment increased the expression of the inflammatory markers ICAM1 and VCAM1 (adhesion molecules) in HUVECs. Treatment with ixmyelocel-T decreased the expression of ICAM1 and VCAM1. Treatment with BMMNCs did not affect the expression of ICAM1 or VCAM1 in the TNFα treated HUVECs (FIG. 31). Another marker of inflammation, MCP-1, was also analyzed by RT-PCR and ELISA in HUVECs that were stimulated with TNFα and co-cultured with ixmyelocel-T or BMMNCs. TNFα treatment increased the expression of MCP-1 in HUVECs, as well as its secretion. Treatment with ixmyelocel-T decreased the expression and secretion of MCP-1, whereas treatment with BMMNCs did not (11983±5357 vs. 23312±11044 pg/mL, p<0.05) (FIG. 32). IL-10 secretion was analyzed by ELISA. Co-culture of TNFα pretreated HUVECs with ixmyelocel-T resulted in IL-10 secretion, which may protect the endothelium by down regulating inflammation (FIG. 33). ELISA analysis indicated that ixmyelocel-T increased IL-10 secretion (61.3±11.2 vs. 1.2±0.5 pg/mL, p<0.001), whereas treatment with BMMNCs had no effect (FIG. 33). Thus, co-culture of ixmyelocel-T with TNFα stimulated HUVECs decreased markers of inflammation.

### Atherosclerosis and Cardiovascular Disease

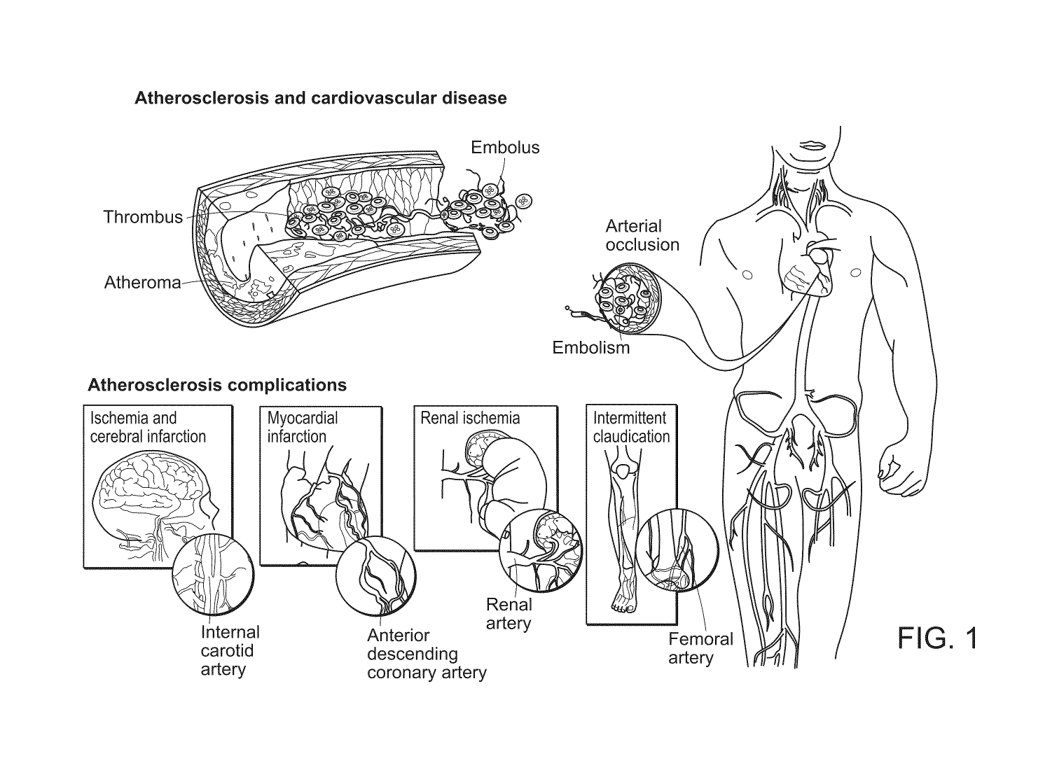

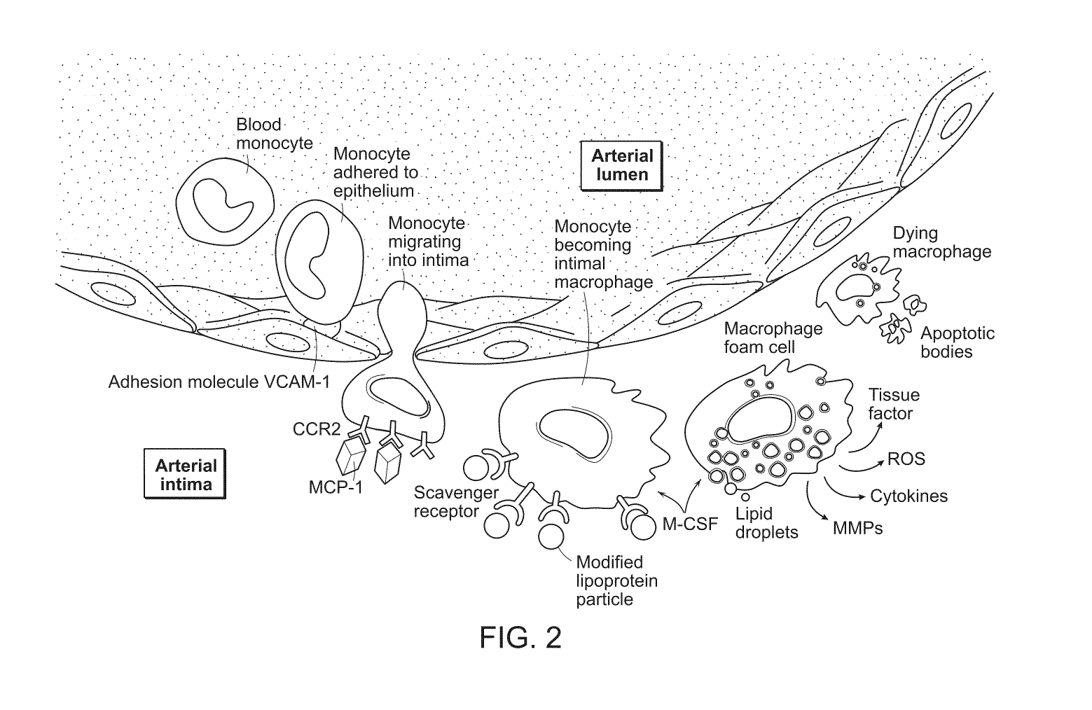

The invention features compositions and methods to treat atherosclerosis and cardiovascular disease. FIG. 1 illustrates formation and complications of atherosclerosis. Exemplary disease states due to atherosclerosis are critical limb ischemia, ischemic dilated cardiomyopathy, cerebral infarction, myocardial infarction, renal ischemia. Atherosclerosis is a complex and multi-factorial disease of the vessel wall involving several different factors, including endothelial dysfunction, chronic inflammation, cellular death, and lipid accumulation. There is a need for a highly efficacious and ideal therapy that addresses all components of this multifactorial disease.

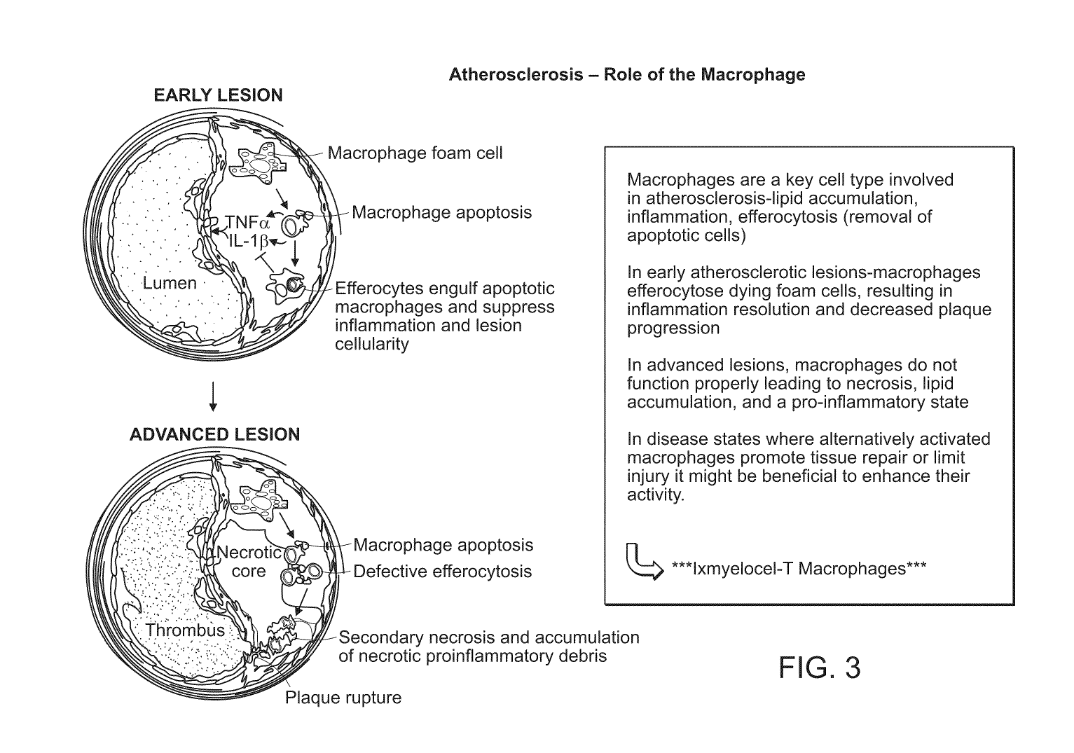

Macrophages are a key cell type involved in atherosclerosis. In particular, macrophages are involved in lipid accumulation, inflammation, and efferocytosis (removal of apoptotic cells). In early atherosclerotic lesions, macrophages efferocytose dying foam cells, resulting in resolution of inflammation and decreased plaque progression. In advanced lesions, macrophages do not function properly, leading to necrosis, lipid accumulation, and a pro-inflammatory state. In disease states where alternatively activated macrophages promote tissue repair or limit injury, it is beneficial to enhance their activity. This invention features macrophages with enhanced activity that promote tissue repair or limit injury (FIG. 3).

### Cholesterol Homeostasis

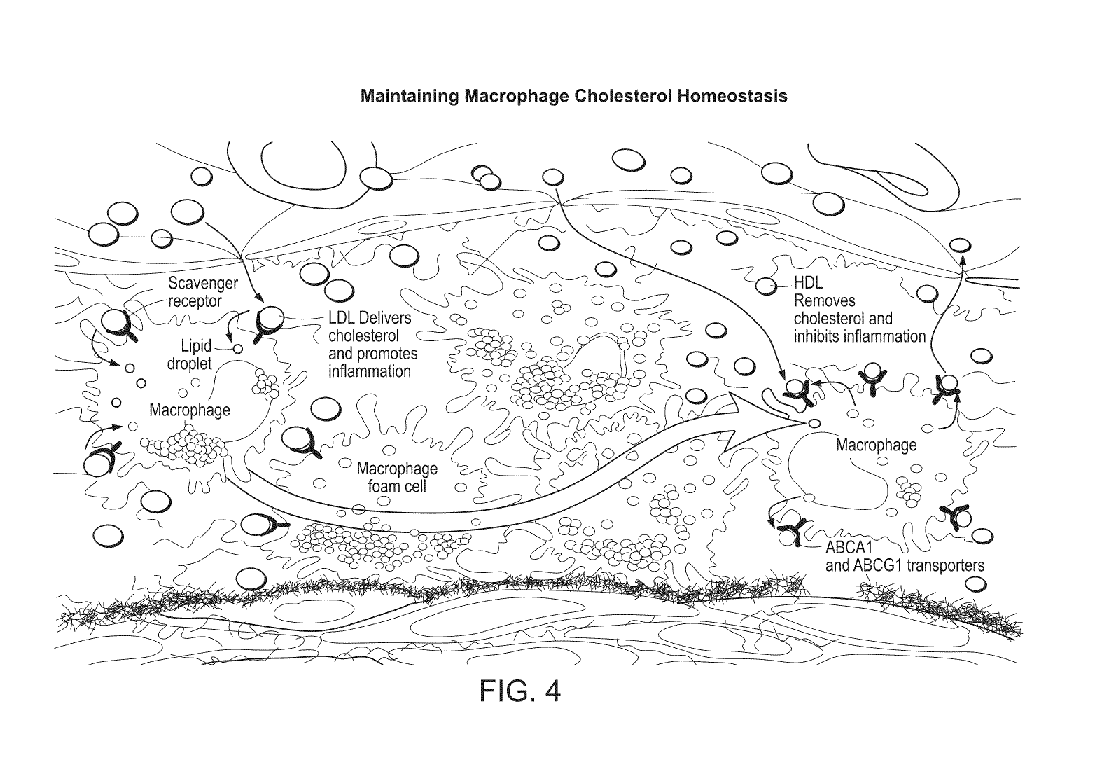

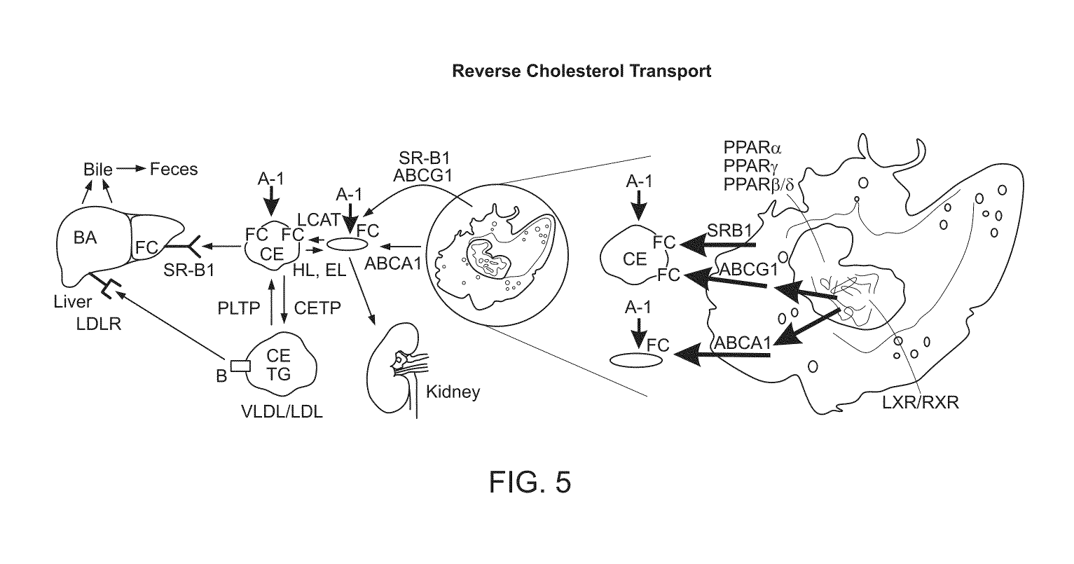

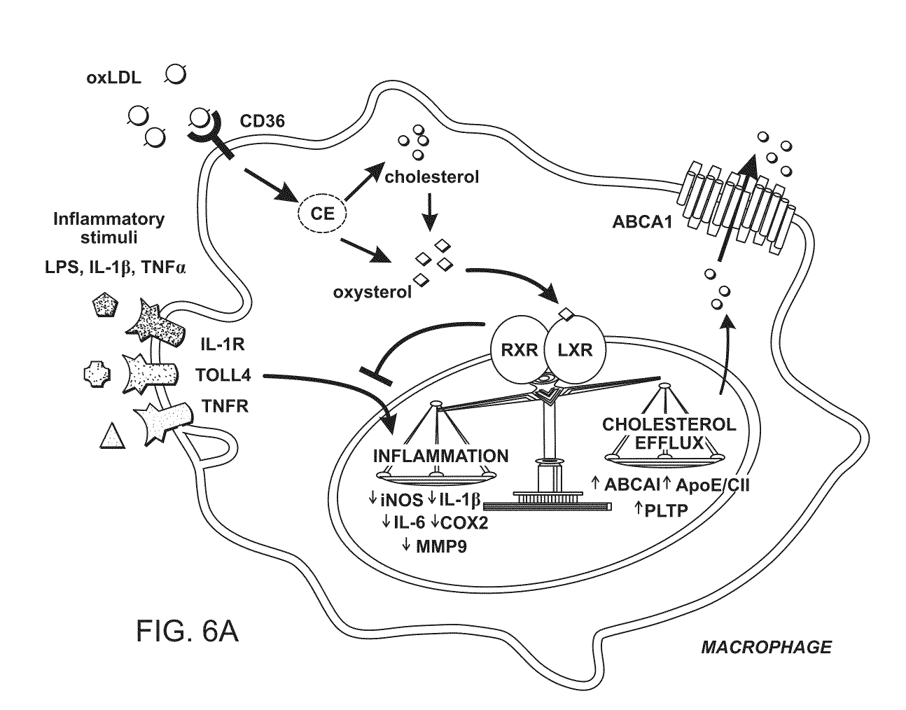

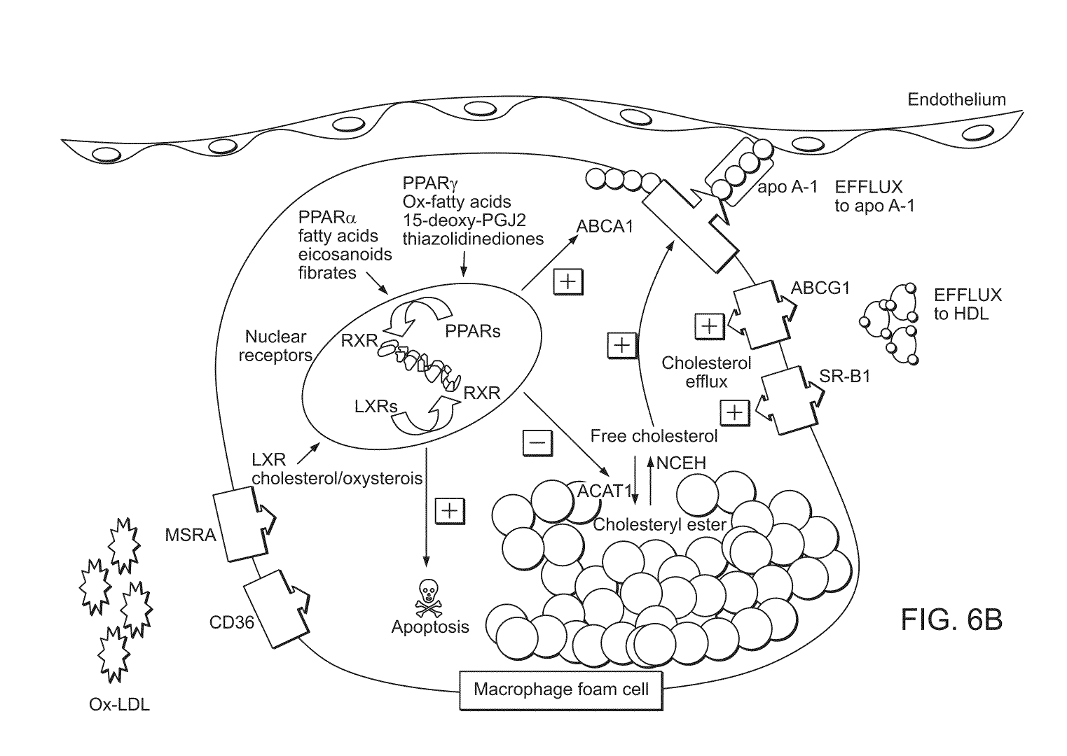

Maintenance of macrophage cholesterol homeostasis (i.e., uptake versus efflux) is essential in preventing the pathogenesis of atherosclerosis. Accumulation of lipid loaded macrophage foam cells is a central feature in the formation of atherosclerosis. An imbalance between cholesterol uptake by scavenger receptors and efflux in macrophages is widely recognized as an underlying mechanism in the progression of atherosclerosis (FIG. 4). Reverse cholesterol transport (RCT) comprises all the different steps in cholesterol metabolism between cholesterol efflux from macrophage foam cells to the final excretion of cholesterol into the feces (either as neutral sterols or after metabolic conversion into bile acids). RCT represents an atheroprotective pathway that is one part of a complex network that determines atherosclerotic lesion formation, progression, and regression (FIG. 5). Macrophages are capable of taking up large quantities of modified cholesterol through scavenger receptors. Macrophages are also capable of disposing of the accumulated cholesterol in a process called cholesterol efflux via cholesterol transporters (ABCA1 and ABCG1). Cholesterol efflux, a first step in RCT, is how macrophages dispose of ingested lipids (e.g. accumulated cholesterol) in order to prevent their death (FIG. 6A-B).

Cholesterol Handling of Ix-MACs

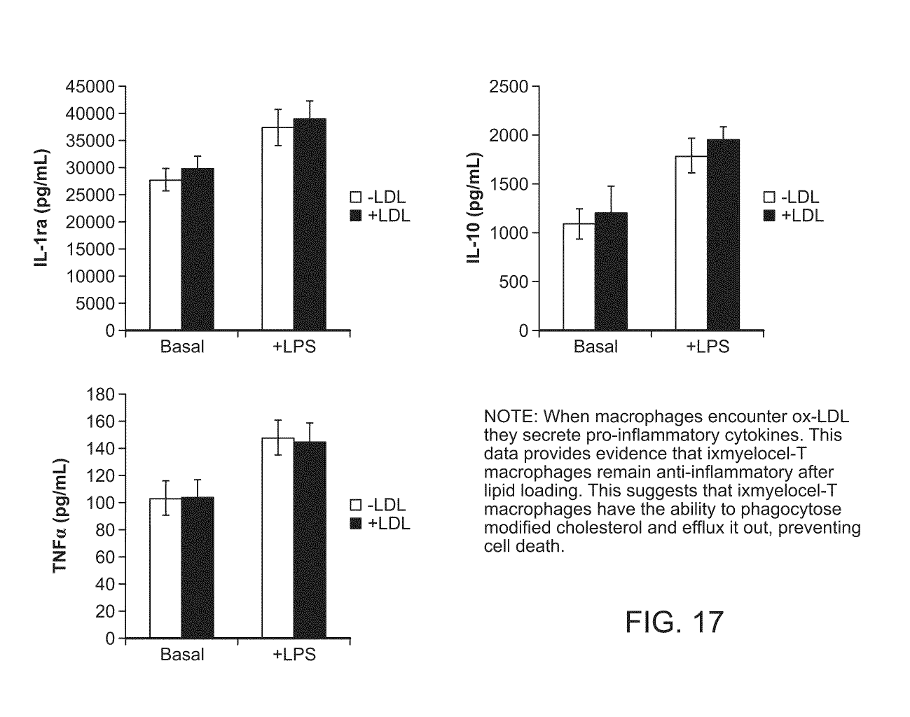

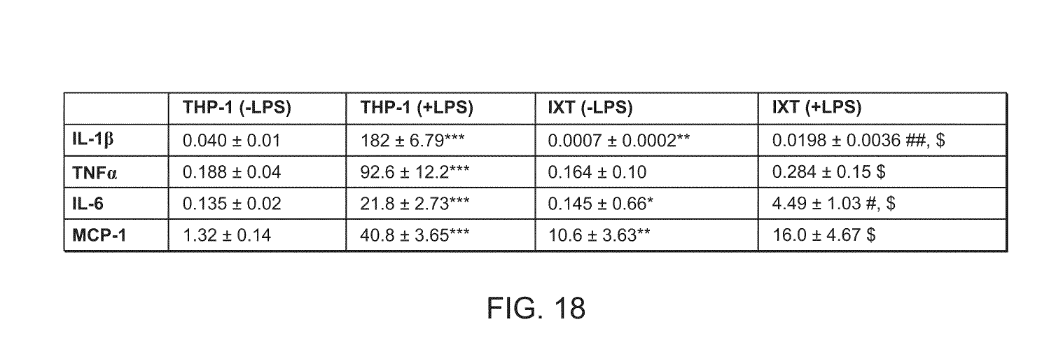

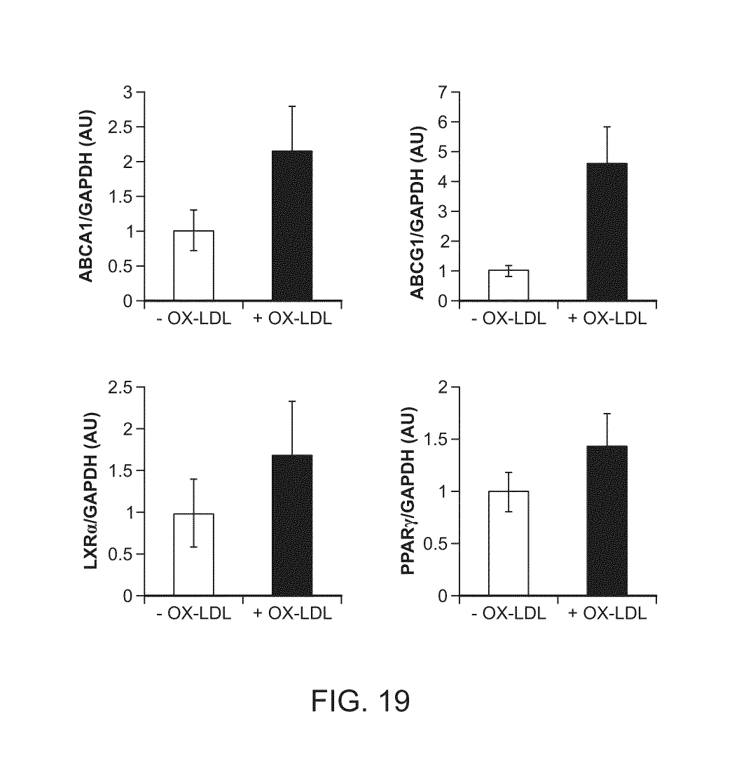

When macrophages are unable to maintain cholesterol homeostasis due to ineffective cholesterol efflux this results in the generation of a pro-inflammatory response. As shown in FIGS. 17 and 18, Ix-MACs, unlike traditional macrophages, which secrete pro-inflammatory cytokines, remain anti-inflammatory after lipid loading. Cholesterol efflux allows macrophages to dispose of accumulated cholesterol. This mechanism involves shuttling cholesterol with several cholesterol transporters, including ABCA1 and ABCG1. As shown in FIG. 19, Ix-MACs treated with oxidized LDL up-regulate cholesterol transport genes ABCA1 and ABCG1. They also up regulate two nuclear receptors involved in cholesterol efflux. This data, combined with the finding that Ix-MACs remain anti-inflammatory after lipid loading, provide evidence that they have the ability handle cholesterol loading efficiently.

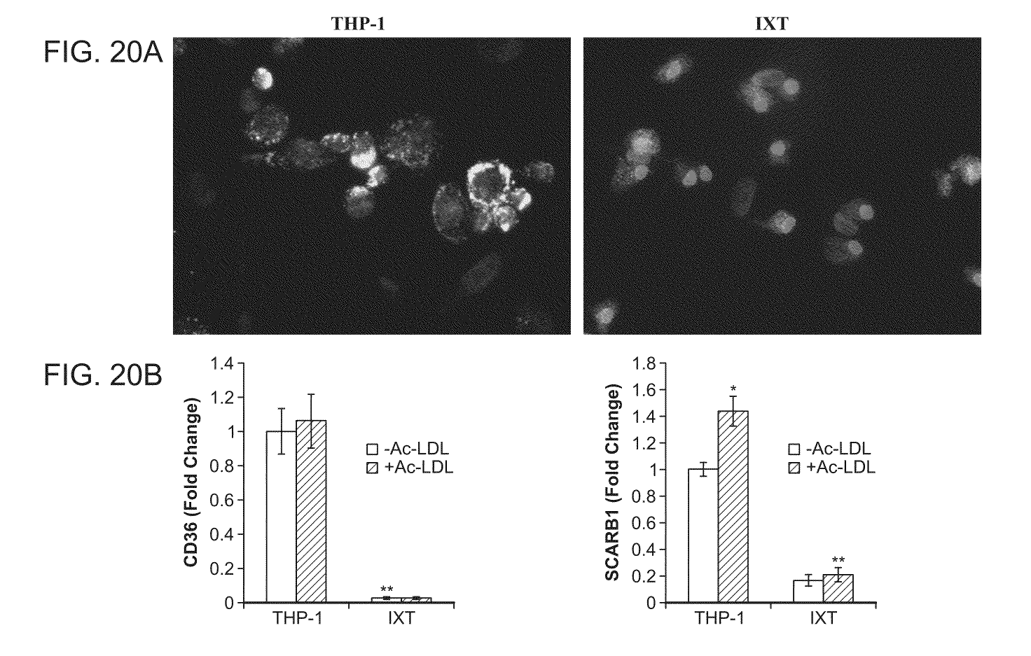

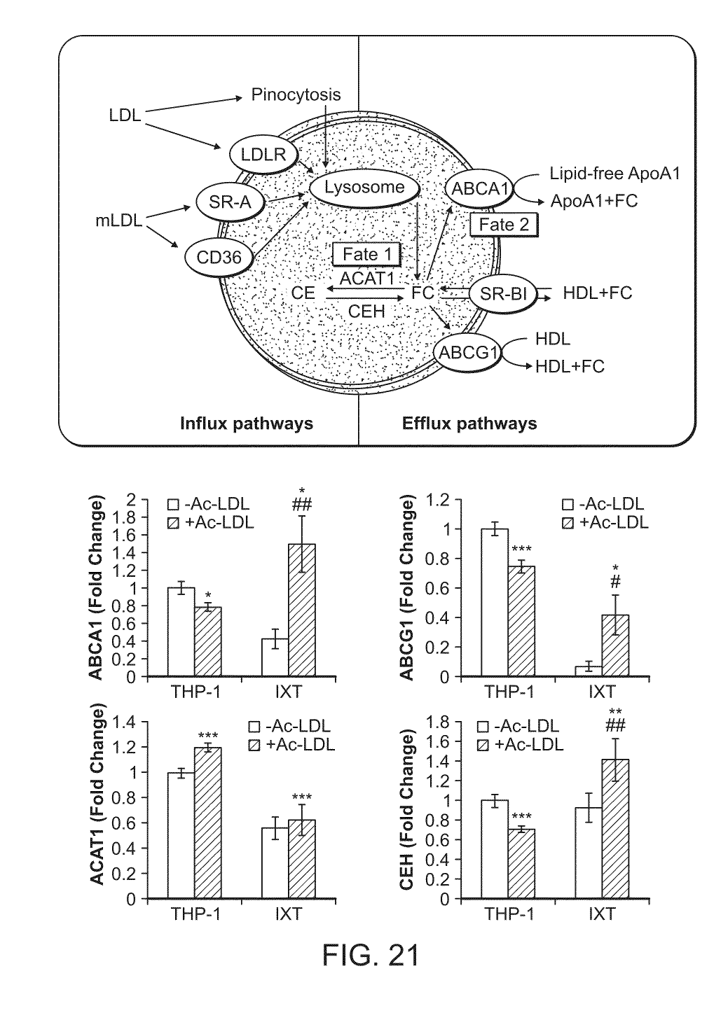

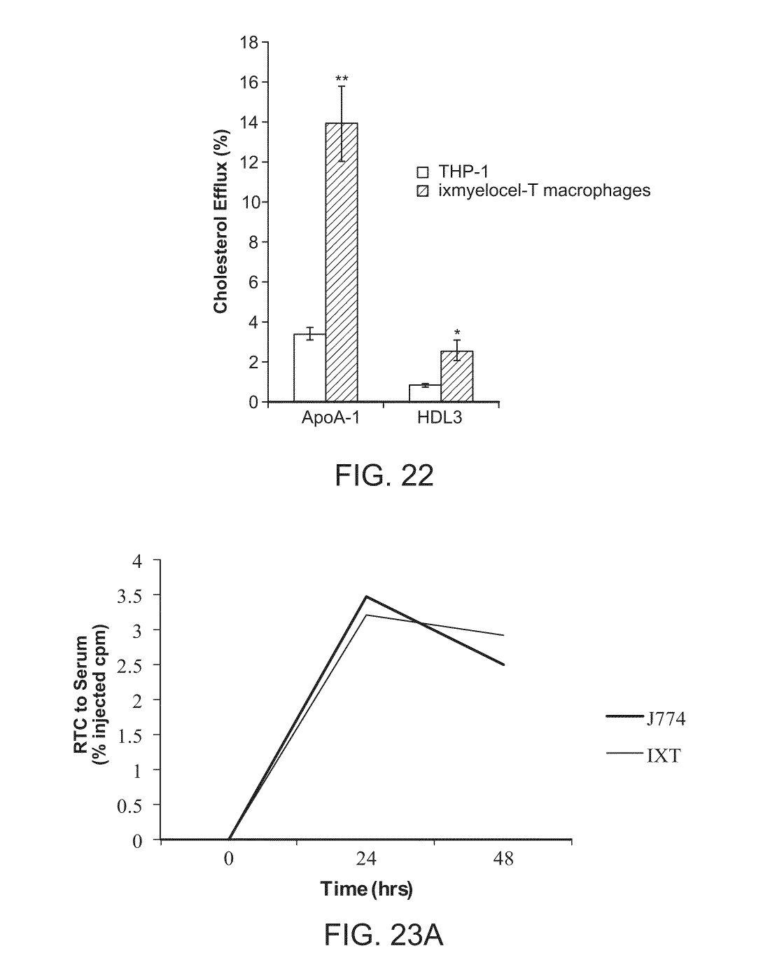

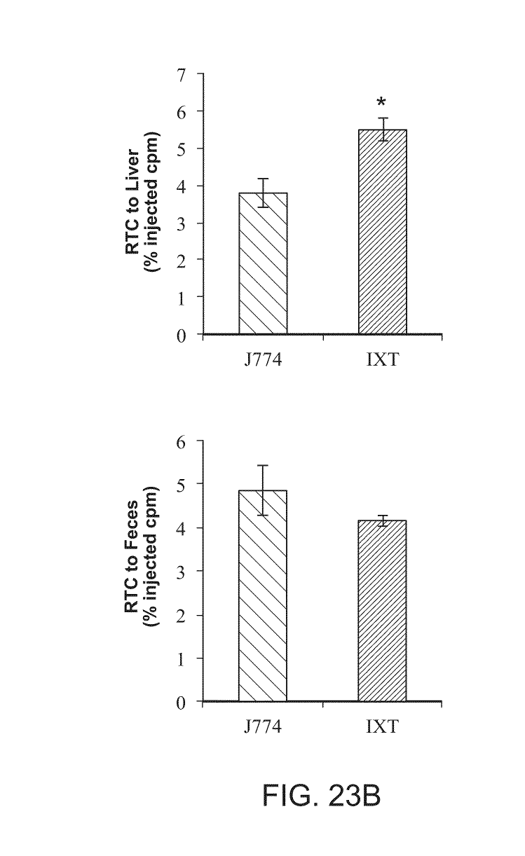

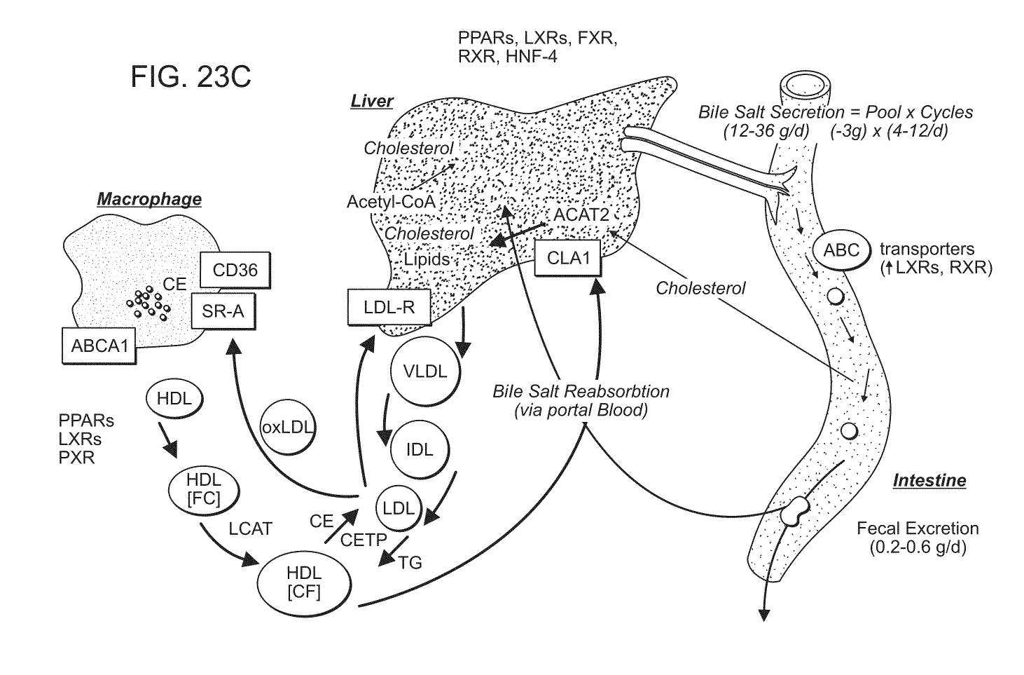

In addition, Ix-MACs have been shown to have reduced scavenger receptor expression, which means the Ix-MACs are less likely to become overladen with modified lipids (FIG. 20). Ix-MACs also display enhanced cholesterol efflux capacity in the expression of cholesterol transport genes (FIG. 21) and using an in vitro cholesterol efflux assays (FIG. 22). Ix-MACs also efflux cholesterol in vivo (FIG. 23). These results indicate that the Ix-MACs have the ability to phagocytose modified cholesterol and efflux it out, preventing cell death.

### Effects of Ixmyelocel-T Cells on Nitric Oxide and Enos

Nitric oxide is essential in vascular repair in response to ischemic injury, suggesting beneficial effects in the treatment of cardiovascular disease Endothelial nitric oxide synthase (eNOS) catalyzes the production of nitric oxide. Treatment with ixmyelocel-T increases plasma nitrate levels and decreases plasma lipid peroxidation, suggesting a preservation of nitric oxide availability and decrease in oxidative stress.

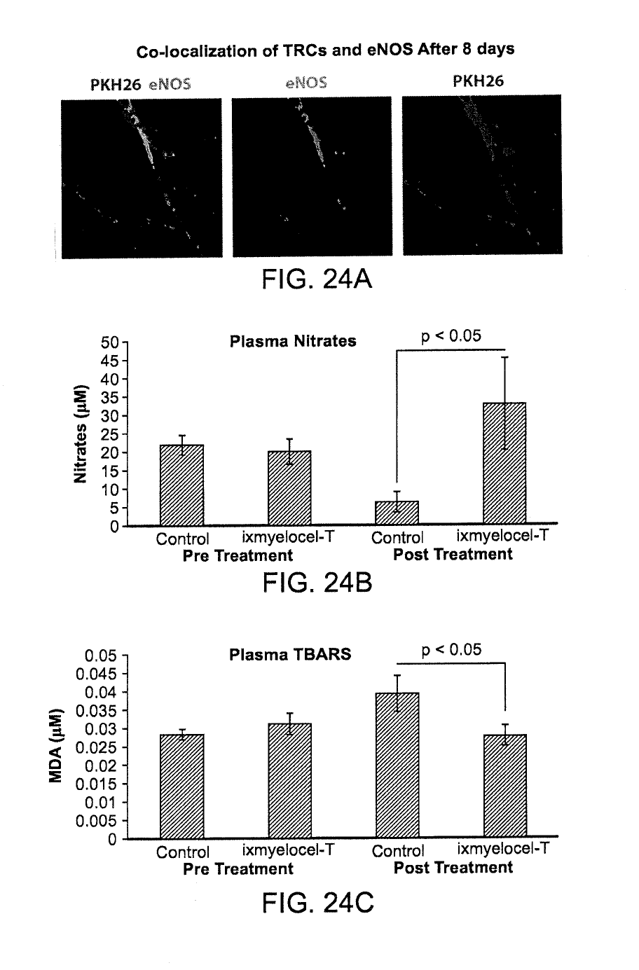

The effect of ixmyelocel-T treatment on plasma nitrates was examined in a rat model of hindlimb ischemia (FIG. 24). Ixmyelocel-T treatment resulted in increased plasma nitrates and decreased in plasma TBARS, suggesting a systemic effect of preservation of the endothelium. eNOS plays a critical role in maintaining vascular homeostasis by exerting anti-inflammatory effects and promoting endothelial repair. In a rat model of hindlimb ischemia, PKH-labeled ixmyelocel-T co-localized with eNOS. Ixmyelocel-T treated rats exhibited increased plasma nitrate levels and decreased plasma lipid peroxidation compared to their vehicle controls; suggesting a preservation of nitric oxide bioavailability and a decrease in oxidative stress.

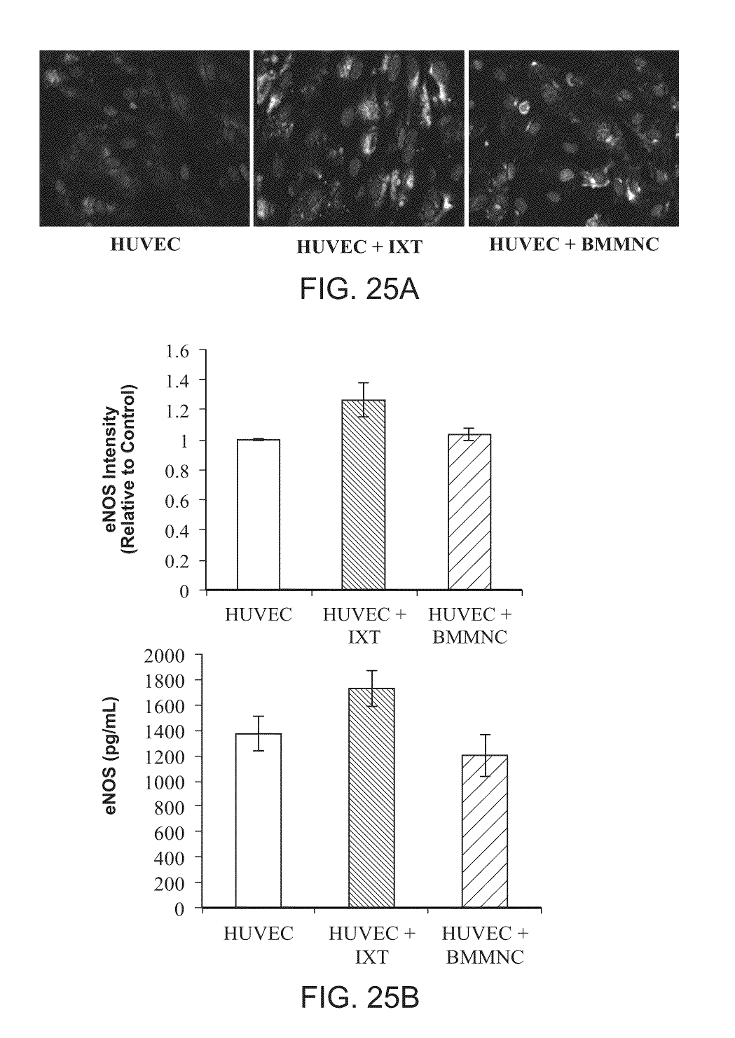

Effect of ixmyelocel-T on eNOS levels was also examined by coculturing ixmyelocel-T or BMMNCs with human umbilical vein endothelial cells (HUVECs) in non-contacting Transwell inserts. HUVECs were co-cultured with ixmyelocel-T and BMMNCs for 2 hours, after which eNOS expression was examined. Immunofluorescence of eNOS was significantly greater in HUVECs co-cultured with ixmyelocel-T compared to control. Co-culture with BMMNCs did not have an effect on HUVEC eNOS immunofluorescence. Co-culture with ixmyelocel-T resulted in increased eNOS (1730±141, vs. 1371±135 pg/mL, p<0.05) in HUVECs measured by ELISA. (FIG. 25). Thus, intracellular levels of eNOS measured by ELISA were also significantly greater in HUVECs co-cultured with ixmyelocel-T compared to control. Co-culture of HUVECs with BMMNC didn't have an effect on intracellular eNOS levels.

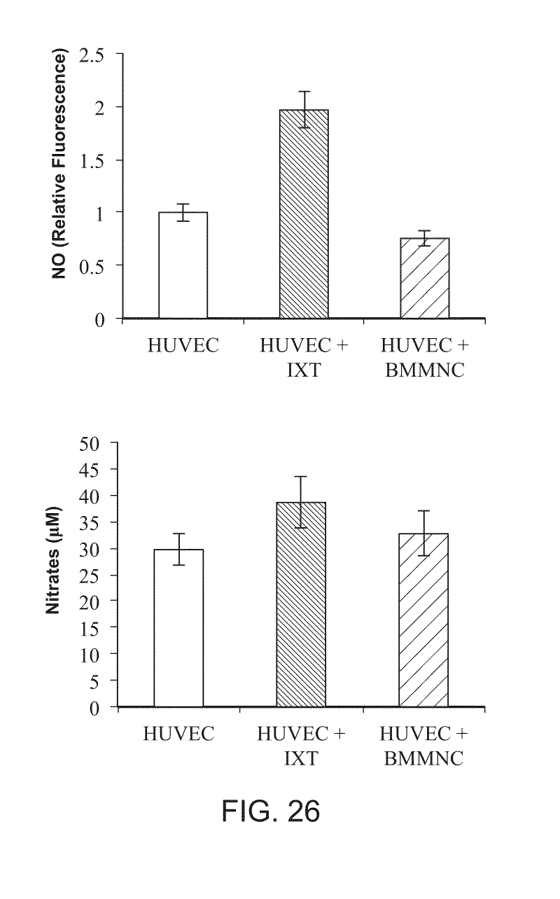

Effect of ixmyelocel-T on NO (an essential molecule involved in vascular repair in response to ischemic injury) levels was also examined by coculturing ixmyelocel-T or BMMNCs with human umbilical vein endothelial cells (HUVECs) in non-contacting Transwell inserts. Co-culture with ixmyelocel-T also resulted in nitric oxide (NO) production (1.97±0.2, vs. 1±0.1 relative fluorescence, p<0.001) measured by DAF-2DA (FIG. 26). Nitric oxide production was measured with the NO probe DAF-2DA. Thus, HUVECs co-cultured with ixmyelocel-T displayed significantly increased nitric oxide production compared to control. BMMNCs did not have an effect on NO production in HUVECs. Nitrates were also measured in the supernatants of the co-cultured cells as a marker of NO production. HUVECs co-cultured with ixmyelocel-T had significantly increased levels of nitrates, whereas co-culture with BMMNCs did not have an effect on nitrates in the HUVECs supernatants.

Effect of Ixmyelocel-T Cells on Reactive Oxygen Species

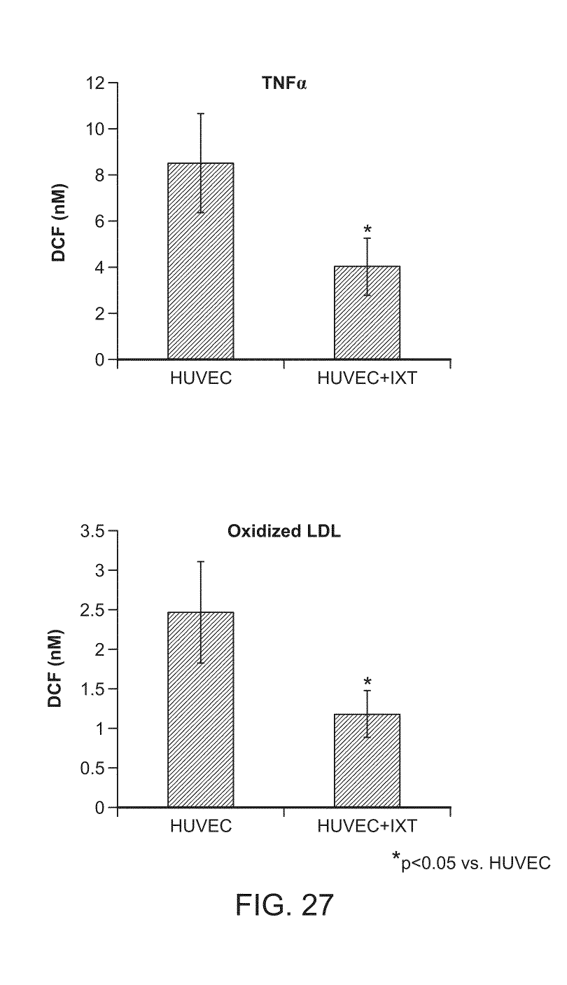

The effect of ixmyelocel-T cells on reactive oxygen species (ROS) levels was also examined. The availability of nitric oxide depends on the balance between its production and inactivation by reactive oxygen species. To determine if ixmyelocel-T protects from oxidative stress, intracellular ROS was measured in TNFα and oxidized LDL stimulated HUVECs co-cultured with ixmyelocel-T. ROS was measured with the fluorescent probe DCFH-DA. Ixmyelocel-T therapy significantly reduced reactive oxygen species (ROS) (FIG. 27). Thus, ixmyelocel-T therapy exerted protective effects on endothelial cells (HUVECs) through down regulation of ROS (FIG. 27), and leads to beneficial effects against cardiovascular diseases.

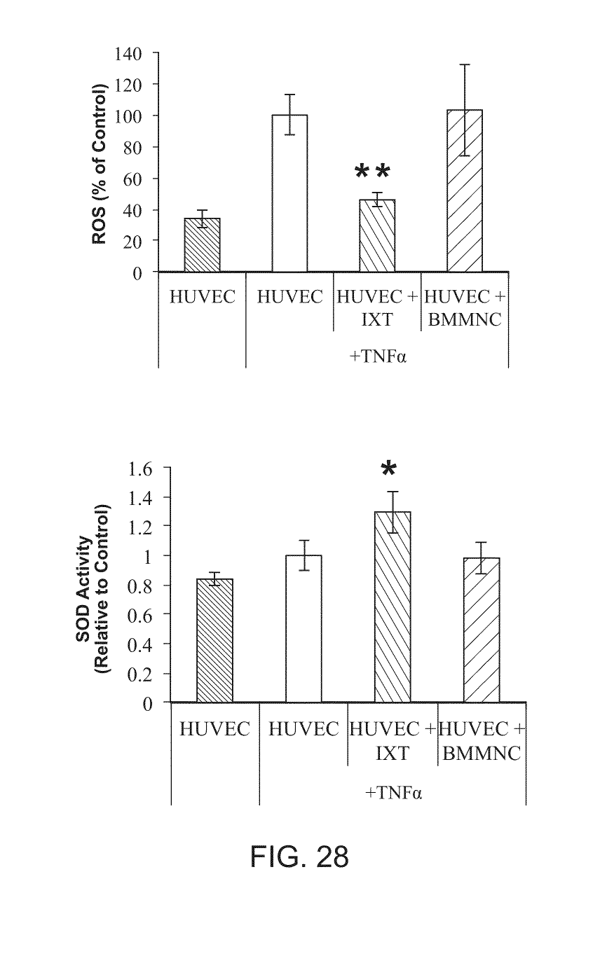

The effect of ixmyelocel-T versus BMMNCs co-culture on ROS and superoxide dismutase (SOD) levels in HUVECs was also determined. Co-culture with ixmyelocel-T significantly reduced the TNFα induced ROS in HUVECs. Ixmyelocel-T decreased the generation of reactive oxygen species (46±4 vs. 100±3% of HUVEC, p<0.01) measured with DCFH-DA. Co-culture of TNFα stimulated HUVECs with BMMNCs did not decrease ROS concentration. Additionally, ixmyelocel-T treatment significantly increased the activity of the antioxidant enzyme SOD in TNFα stimulated HUVECs (1.3±0.1, vs. 1±0.1% of HUVEC, p<0.05). In contrast, co-culture with BMMNCs did not increase SOD activity in the TNFα stimulated HUVECs. Thus, ixmyelocel-T decreased TNFα mediated oxidative stress and increased SOD activity in co-cultured HUVECs (FIG. 28).

Effect of Ix-MACs and Ixmyelocel-T Cells on Apoptotic or Necrotic Tissue

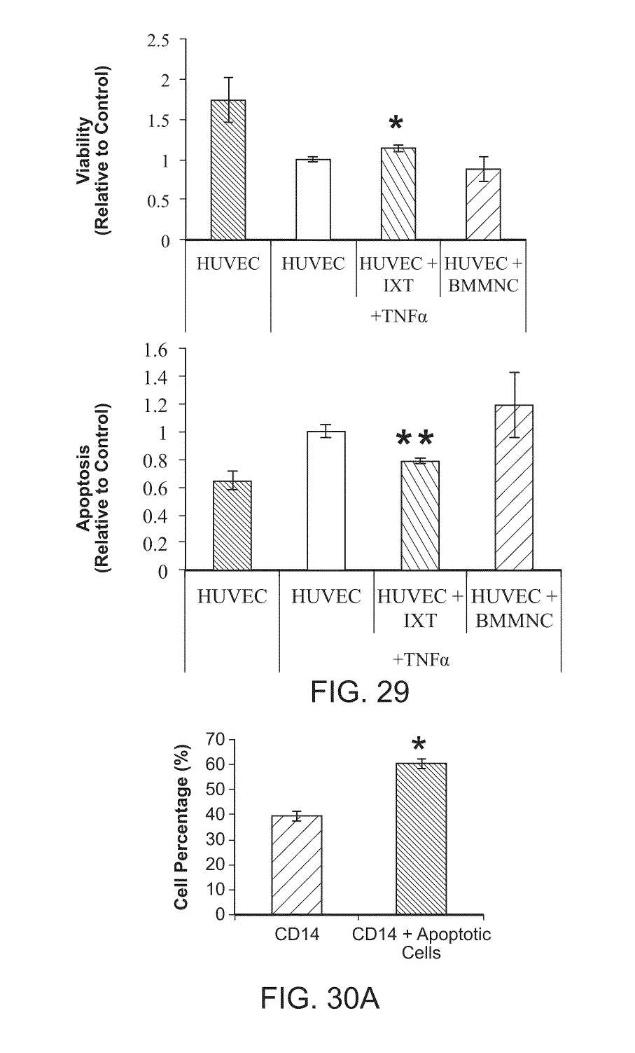

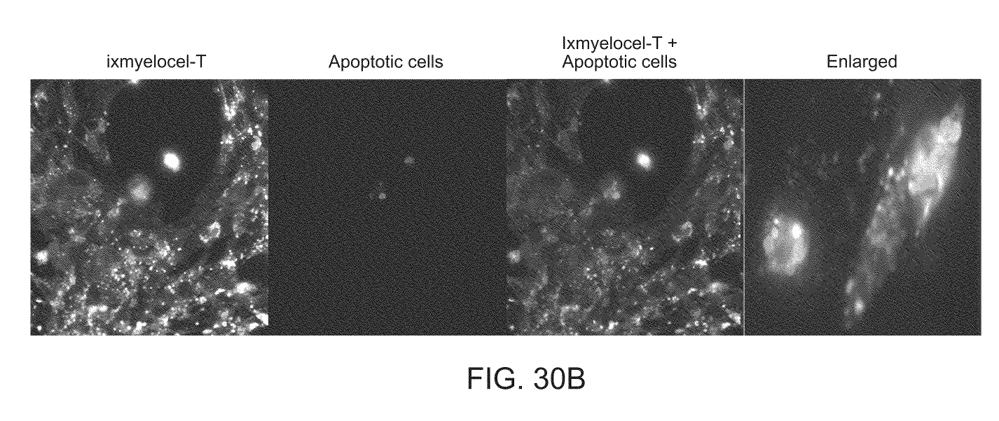

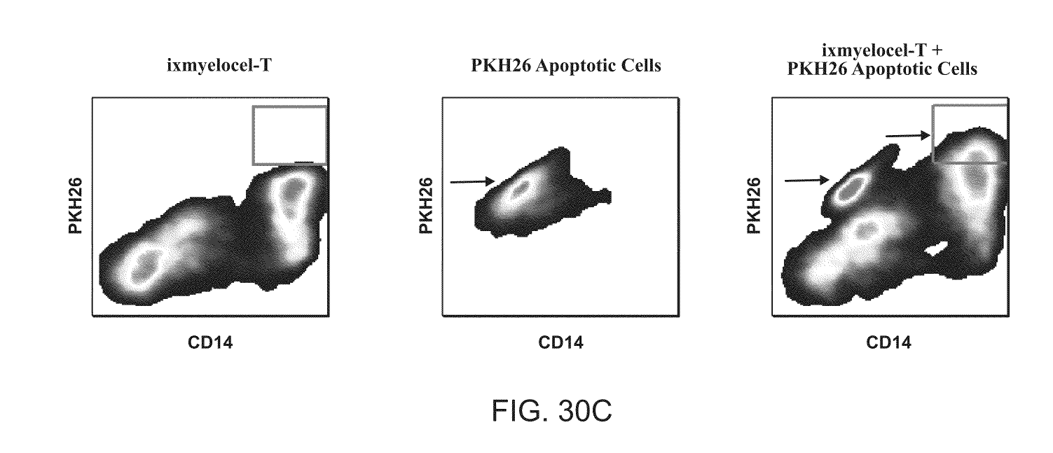

The effect of Ix-MACs and ixmyelocel-T cells on removal of apoptotic or necrotic tissue was examined. Ixmyelocel-T decreased TNFα induced endothelial cell apoptosis. Apoptosis analyzed by a caspase 3/7 assay demonstrated that ixmyelocel-T decreased apoptosis in TNFα treated HUVECs (0.78±0.02, vs. 1±0.05 relative to HUVEC, p<0.001) (FIG. 29). Co-culture with BMMNCs had no effect on HUVEC apoptosis. In addition, in the process of efferocytosis, ixmyelocel-T alternatively activated macrophages (Ix-MACs) readily phagocytozed apoptotic cells (FIG. 30). Efferocytosis was measured by microscopy and flow cytometry. 60% of ixmyelocel-T CD14+ cells efferocytosed apoptotic cells (n>5). *P<0.001 vs. CD14. Magnification: 60×. Thus, ixmyelocel-T decrease TNFα induced endothelial cell apoptosis and remove apoptotic/necrotic tissue. In summary, ixmyelocel-T stimulated NO production, reduced oxidative stress and inflammation, and prevented apoptosis in endothelial cells. BMMNCs did not exhibit similar results. This is most likely due to the anti-inflammatory cell phenotypes associated with ixmyelocel-T's expansion process. This study indicates that ixmyelocel-T and IxMACs are superior to BMMNCs in the treatment of diseases associated with endothelial dysfunction and vascular inflammation.

Collectively, the data described above shows that ixmyelocel-T and Ix-MACs therapy is beneficial for the treatment of atherosclerosis and cardiovascular diseases. Ix-MACs play an immunomodulatory role in anti-inflammatory cytokine secretion. Ix-MACs also contribute to tissue remodeling and phagocytosis of necrotic/apoptotic tissue. Finally, Ix-MACs also have modified cholesterol uptake and efflux. In particular, Ix-MACs have enhanced cholesterol uptake that can protect the vasculature by removing atherogenic lipoproteins which elicit strong pro-inflammatory responses. Cholesterol efflux also allows cholesterol to be disposed of, preventing increased inflammation and cell death. Thus, Ix-MACs address many of the components of the multi-factorial cardiovascular disease, making Ix-MACs not only an ideal and highly efficacious therapy.

Ix-MACs and Ixmyelocel-T cell compositions are useful for a variety of anti-inflammatory therapeutic methods including cardiovascular disease, such as atherosclerosis and ischemic conditions. Ischemic conditions include, but are not limited to, limb ischemia, congestive heart failure, cardiac ischemia, kidney ischemia and ESRD, stroke, and ischemia of the eye.

For example, the Ix-MACs and Ixmyelocel-T cell compositions are useful in modulating cholesterol efflux, decreasing atherosclerotic lesions, decreasing oxidative stress of a tissue such as the endothelium, increasing plasma nitrate levels, decreasing plasma lipid peroxidation, increasing the expression of endothelial nitric oxide synthase (eNOS), and increasing nitric oxide production (NO) in a cell.

Additionally, the Ix-MACs are useful in tissue regeneration or repair, treating ischemic tissues, and inducing angiogenesis.

Ix-MACs and Ixmyelocel-T cell compositions are administered to mammalian subjects, e.g., human, to effect a therapeutic benefit. The Ix-MACs and Ixmyelocel-T cell compositions are administered allogeneically or autogeneically.

The described Ix-MACs and Ixmyelocel-T cell compositions can be administered as a pharmaceutically or physiologically acceptable preparation or composition containing a physiologically acceptable carrier, excipient, or diluent, and administered to the tissues of the recipient organism of interest, including humans and non-human animals. Ix-MACs and ixmyelocel-T containing compositions can be prepared by resuspending the cells in a suitable liquid or solution such as sterile physiological saline or other physiologically acceptable injectable aqueous liquids. The amounts of the components to be used in such compositions can be routinely determined by those having skill in the art.

The Ix-MACs and ixmyelocel-T cell compositions thereof can be administered by placement of the cell suspensions onto absorbent or adherent material, i.e., a collagen sponge matrix, and insertion of the Ix-MACs and ixmyelocel-T-containing material into or onto the site of interest. Alternatively, the Ix-MACs and ixmyelocel-T cell compositions can be administered by parenteral routes of injection, including subcutaneous, intravenous, intramuscular, and intrasternal. Other modes of administration include, but are not limited to, intranasal, intrathecal, intracutaneous, percutaneous, enteral, and sublingual. In one embodiment of the present invention, administration of the Ix-MACs and ixmyelocel-T cell compositions can be mediated by endoscopic surgery.

For injectable administration, the composition is in sterile solution or suspension or can be resuspended in pharmaceutically- and physiologically-acceptable aqueous or oleaginous vehicles, which may contain preservatives, stabilizers, and material for rendering the solution or suspension isotonic with body fluids (i.e. blood) of the recipient. Non-limiting examples of excipients suitable for use include water, phosphate buffered saline, pH 7.4, 0.15 M aqueous sodium chloride solution, dextrose, glycerol, dilute ethanol, and the like, and mixtures thereof. Illustrative stabilizers are polyethylene glycol, proteins, saccharides, amino acids, inorganic acids, and organic acids, which may be used either on their own or as admixtures. The amounts or quantities, as well as the routes of administration used, are determined on an individual basis, and correspond to the amounts used in similar types of applications or indications known to those of skill in the art.

Consistent with the present invention, the Ix-MACs and ixmyelocel-T cell compositions can be administered to body tissues, including liver, pancreas, lung, salivary gland, blood vessel, bone, skin, cartilage, tendon, ligament, brain, hair, kidney, muscle, cardiac muscle, nerve, skeletal muscle, joints, and limb.

The number of cells in an Ix-MAC suspension and the mode of administration may vary depending on the site and condition being treated. As non-limiting examples, in accordance with the present invention, about 40-200×10⁶ Ix-MACs are injected to effect a therapeutic benefit. A skilled practitioner can modulate the amounts and methods of Ix-MAC -based treatments according to requirements, limitations, and/or optimizations determined for each case.

We claim:

1. A composition comprising a population of cells of hematopoietic lineage, wherein the composition contains CD14⁺ macrophages, and wherein when the cells are contacted with a pro-inflammatory stimulus produce inflammatory cytokines such that the anti-inflammatory cytokine: pro-inflammatory cytokine ratio produced is at least 2:1.

2. The composition of claim 1, wherein the composition further comprises CD14⁺ monocytes.

3. The composition of claim 1, wherein the ratio is at least 5:1, 10:1, 25:1, 50:1 or 100:1.

4. The composition of claim 1, wherein the cells are derived from bone marrow, peripheral blood, umbilical cord blood, fetal liver, human embryonic stem cells (huES), induce pluripotent stem cells (iPS) or parthenogenetic cells.

5. The composition of claim 1, wherein the composition has one or more of the following characteristics:
a) the viability of the cells is at least 75%;
b) contains less than 2 μg/ml serum albumin;
c) substantially free of horse serum or
d) substantially free of mycoplasm, endotoxin and microbial contamination.

6. The composition of claim 1, wherein the cells are in a pharmaceutical-grade electrolyte solution suitable for human administration.

7. The composition of claim 1, wherein the total number of cells is 40 to 200 million.

8. The composition of claim 1, wherein the cells are in a volume less than 15 mLs.

9. The composition of claim 1, wherein the cells produce at least 100 pg per 2×10⁶ cells of one or more anti-inflammatory cytokines.

10. The composition of claim 1, where in the anti-inflammatory cytokine is IL-10 or ILRa.

11. The composition of claim 1, wherein the pro-inflammatory stimulus is lipopolysaccharide (LPS).

12. The composition of claim 1, wherein at least 5% of the CD14⁺ macrophages are autofluorescent.

13. The composition of claim 1, wherein said composition is an in-vitro expanded cell population.

14. The composition of claim 2, wherein the CD14⁺ monocytes are expanded in vitro.

15. The composition of claim 14, wherein the CD14⁺ monocytes differentiate into CD14⁺ macrophages in vitro.

16. The composition of claim 1, wherein the CD14⁺ macrophages are derived from CD34⁺ hematopoietic progenitor cells that have been differentiated in vitro.

17. The composition of claim 16, wherein the CD34+ hematopoietic progenitor are myeloid cells.

18. The composition of claim 17, wherein the myeloid cells are myeolomonocytes.

19. The composition of claim 1, wherein the cells are isolated from an in-vitro expanded cell culture.

20. The composition of claim 19, wherein in-vitro expanded cell culture is derived from mononuclear cells.

21. The composition of claim 19, wherein in-vitro expanded cell culture comprises a mixed population of cells of hematopoietic, mesenchymal and endothelial linage.

22. The composition of claim 19, wherein in-vitro expanded cell culture comprises a mixed population of cells of hematopoietic and mesenchymal linage.

23. The composition of claim 19, wherein in-vitro expanded cell culture comprises a population of hematopoietic cells.

24. The composition of claim 21 or 22, wherein the mixed population of cells are about 5-75% viable CD90⁺ cells with the remaining cells in the composition being CD45⁺.

25. The composition of claim 23, wherein the hematopoietic cells are CD45⁺.

26. The composition of claim 1, wherein at least 5% of the CD14⁺ macrophages are CD66b-negative, CD18⁺, CD33⁺, CD11b⁺, CD11c⁺, CD91-negative, CD141⁺, HLA-DR-negative, CD209-negative, CD16-negative, and/or CD1c-negative.

27. The composition of claim 26, wherein at least 10% of the CD14+ macrophages are CD66b-negative, CD18+, CD33+, CD11b+, CD11c+, CD91-negative, CD141+, HLA-DR-negative, CD209-negative, CD16-negative, and/or CD1c-negative.

28. The composition of claim 27, wherein at least 15% of the CD14+ macrophages are CD66b-negative, CD18+, CD33+, CD11b+, CD91-negative, CD141+, HLA-DR-negative, CD209-negative, CD16-negative, and/or CD1c-negative.

29. The composition of claim 1, wherein at least 5% of the CD14⁺ macrophages express PPARγ, CD206, CD163, CD204, SR-B1, MERTK, and/or TGFβ.

30. The composition of claim 29, wherein at least 10% of the CD14⁺ macrophages express PPARγ, CD206, CD163, CD204, SR-B1, MERTK, and/or TGFβ.

31. The composition of claim 30, wherein at least 15% of the CD14⁺ macrophages express PPARγ, CD206, CD163, CD204, SR-B1, MERTK, and/or TGFβ.

32. The composition of claim 1, wherein the CD14⁺ macrophages express a higher level of PPARγ, CD206, CD163, CD204, SR-B1, MERTK, and/or TGFβ compared to M1 macrophages.

33. The composition of claim 32, wherein the CD14⁺ macrophages express an at least two-fold higher level of PPARγ, CD206, CD 163, CD204, SR-B 1, MERTK, and/or TGF compared to M1 macrophages.

34. The composition of claim 1, wherein the CD14⁺ macrophages express a lower level of CCR7, IL-1B, and/or TNFα compared to M1 macrophages.

35. The composition of claim 34, wherein the CD14⁺ macrophages express an at least two-fold lower level of CCR7, IL-1B, and/or TNFα compared to M1 macrophages.

36. The composition of claim 1, wherein the CD14⁺ macrophages are exposed to a pro-inflammatory stimulus, and wherein the expression level of a pro-inflammatory cytokine in the CD14⁺ macrophages after the exposure is 100% or less of the expression level of the pro-inflammatory cytokine prior to the exposure.

37. The composition of claim 36, wherein the pro-inflammatory stimulus comprises a pathogen, lipopolysaccharide, interferon-gamma, lipoxin, a leukotriene, an endotoxin, or debris from a dead cell.

38. The composition of claim 36, wherein the pro-inflammatory cytokine comprises TNFβ, IL-1A, IL-1B, or IL-12.

39. A method of modulating cholesterol efflux in vascular tissue of a subject comprising administering to a subject in need thereof the composition of claim 1 or a composition comprising ixmyelocel-T.

40. A method of decreasing atherosclerotic lesions in a subject comprising administering to a subject in need thereof the composition of claim 1 or a composition comprising ixmyelocel-T.

41. A method of treating atherosclerosis comprising administering to a subject in need thereof the composition of claim 1 or a composition comprising ixmyelocel-T.

42. A method of decreasing oxidative stress of a tissue comprising contacting the tissue with composition of claim 1 or a composition comprising ixmyelocel-T.

43. The method of claim 42, wherein the tissue is endothelium.

44. A method of increasing plasma nitrate levels and/or decreasing plasma lipid peroxidation in a subject comprising administering to a subject in need thereof the composition of claim 1 or a composition comprising ixmyelocel-T.

45. A method of increasing the expression of endothelial nitric oxide synthase (eNOS) and/or nitric oxide production (NO) in a cell comprising contacting the cell with composition of claim 1 or a composition comprising ixmyelocel-T.

46. A method of tissue regeneration or repair comprising administering to a patient in need thereof the composition of claim 1.

47. A method of treating ischemic disorders comprising administering to a patient in need thereof the composition of claim 1.

48. A method of inducing angiogenesis in a tissue comprising administering to a patient in need thereof the composition of claim 1.
